(19) United States
(12) Patent Application Publication
(10) Pub. No.: US 2005/0191273 A1
(43) Pub. Date: Sep. 1, 2005

(54) Cytokine inhibition of eosinophils

(76) Inventors: Marc Elliot Rothenberg, Cincinnati, OH (US); Patricia Chandhok Fulkerson, Cincinnati, OH (US)

Correspondence Address: WOOD, HERRON & EVANS, LLP, 2700 CAREW TOWER, 441 VINE STREET, CINCINNATI, OH, 45202, US

(21) Appl. No.: 11/091,288

(22) Filed: Mar. 28, 2005

Related U.S. Application Data
(63) Continuation-in-part of application No. 10/752,659, filed on Jan. 7, 2004.
(60) Provisional application No. 60/438,412, filed on Jan. 7, 2003.

(51) Int. Cl. A61K 38/19

(52) U.S. Cl. 424/85.1

(57) ABSTRACT

The cytokine CXCL9 (MIG) inhibited eosinophil responses by a CCR3- and Rac2-dependent mechanism.

### RELATED APPLICATION

This application is a Continuation-in-Part of U.S. patent application Ser. No. 10/752,659 filed Jan. 7, 2004, now pending, which claims priority to U.S. Provisional Patent Application Ser. No. 60/438,412 filed Jan. 7, 2003, each of which is expressly incorporated by reference herein in its entirety.

The U.S. Government has a paid-up license in this invention and the right in limited circumstances to require the patent owner to license others on reasonable terms as provided for by the terms of Grant Nos. NIH R01 AI42242, R01 AI45898, P01 HL076383 awarded by the National Institutes of Health.

### FIELD OF THE INVENTION

The invention is directed to compositions and methods of chemoattractant-induced alteration of eosinophil function and distribution.

### BACKGROUND

Eosinophils are one type of granulocytic leukocyte (white blood cell) or granulocyte that normally appears in the peripheral blood at a concentration of about 1-3% of total leukocytes. Their presence in tissues is normally primarily restricted to the gastrointestinal mucosa. In various disease states, eosinophils are increased in the peripheral blood and/or tissues, a condition termed eosinophilia and described by Rothenberg in Eosinophilia, N. Engl. J. Med. 338, 1592-1600 (1998).

Eosinophil accumulation in the peripheral blood and tissues is a hallmark feature of several diseases. These diseases include atopic diseases (allergic disorders such as allergic rhinitis, asthma, and eczema); parasitic infections; certain types of malignancies; chronic inflammatory disorders such as inflammatory bowel disease; and specific syndromes such as eosinophilic gastroenteritis, eosinophilic colitis, eosinophilic cellulitis, eosinophilic esophagitis, eosinophilic fascitis; and systemic diseases such as Churg Strauss syndrome, eosinophilic pneumonia, and the idiopathic hypereosinophilic syndrome. Eosinophil accumulation in tissues may cause potent pro-inflammatory effects or tissue remodeling. Inappropriate accumulation and activation of eosinophils can result in the release of a range of inflammatory mediators, including toxic granule proteins, cytokines, and activated oxygen species that contribute to disease pathogenesis.

Numerous mediators have been identified as eosinophil chemoattractants. These include diverse molecules such as lipid mediators (platelet activating factor (PAF), leukotrienes) and recently chemokines, such as the eotaxin subfamily of chemokines. Chemokines are small secreted proteins produced by tissue cells and leukocytes that regulate leukocyte homing during homeostatic and inflammatory states. Two main subfamilies (CXC and CC chemokines) are distinguished depending upon the arrangement of the first two cysteines, which are separated by one amino acid (CXC) or are adjacent (CC).

In the asthmatic lung, eosinophil recruitment is regulated by integrated signaling events between chemokines that operate through chemokine receptor 3 (CCR3), lipid mediators (e.g. platelet activating factor (PAF) and leukotrienes), enzymes (e.g. acidic mammalian chitinase) and the Th2 cytokines IL-4, IL-5, and IL-13. IL-5 is critical for the induction of peripheral blood eosinophilia, and also acts in synergy with the eosinophil-specific chemokines eotaxin-1 and eotaxin-2 (CCR3 ligands), induced by IL-4 and IL-13, to coordinate tissue accumulation of eosinophils. Following CCR3 binding, the eotaxins activate numerous events including actin reorganization, calcium mobilization, and production of reactive oxygen species. In addition, the eotaxins couple to signaling pathways that have been demonstrated to mediate migration, including the mitogen-activated protein kinase family and the Rho family of small GTPases. In neutrophils, Rho GTPases (e.g. Rho, Rac and Cdc42) control a wide spectrum of cellular functions, including cytoskeletal organization, transcription, superoxide production and cell growth and proliferation; however, their role in eosinophils has not been established.

Chemokines are the largest family of cytokines in the human genome. They have an essential role in diverse processes including inflammation, organ development, tumor cell growth and metastasis, atherosclerosis, and angiogenesis. More than 45 chemokines and 16 seven-transmembrane spanning-G-protein-coupled receptors (most being promiscuous) have been characterized, and the individual and cooperative interaction of individual chemokines and their receptors is being evaluated. The role of naturally occurring chemokine antagonists is also being evaluated; at least eight different chemokines that act as natural antagonists have been described. In addition to being agonists for their cognate receptors, these chemokines induce inhibitory activity by acting as competitive antagonists for a variety of other receptors. For example, while MCP-3 is normally a ligand for CCR2, it is a competitive antagonist for CCR5. Indeed, numerous microbial pathogens have exploited chemokine receptors by encoding for chemokine homologues that have antagonistic properties. Initial mechanistic analysis has focused primarily on receptor blockade, but for several of these natural antagonists the pronounced inhibitory activity cannot be explained by competitive receptor binding alone. There is little insight into the mechanisms that regulate the inhibitory activity of natural chemokines except for models that rely on simple receptor antagonism. It was recently proposed that diverse signaling events can be induced by ligands that bind to the same receptor.

The finding that eosinophils normally account for only a small percentage of circulating or tissue dwelling cells, and that their numbers markedly and selectively increase under specific disease states, indicates the existence of molecular mechanisms that regulate the selective generation and accumulation of these leukocytes. A composition to regulate eosinophil function would therefore be desirable, in view of the wide variety of eosinophil-mediated conditions. For example, pediatric asthma is an eosinophil-mediated condition whose incidence is on the rise and is now the chief diagnosis responsible for pediatric hospital admissions. Alleviation of asthma, along with the spectrum of other eosinophil-mediated conditions, by altering eosinophil function would be of benefit.

### SUMMARY OF THE INVENTION

One embodiment of the invention is directed to a method of inhibiting eosinophil function. A pharmaceutical composition containing an isolated cytokine with eosinophil function-inhibitory activity is administered to a patient in a pharmaceutically effective amount to inhibit eosinophil function. The composition may inhibit receptor expression, receptor internalization, signal transduction, transmigration, desensitization, degranulation, and/or mediator release. The cytokine may be monokine induced by interferon γ (MIG), and/or IFN-γ-inducible protein of 10 kDa (IP-10).

Another embodiment of the invention is a method of reducing allergen-induced eosinophilia, for example, in an airway, the lungs, the trachea, the bronchoalveolar lavage fluid, or the blood. Eosinophilia may also be reduced in a body part affected by an allergy, such as eyes, skin, and gut.

Another embodiment of the invention is a treatment method by administering a pharmaceutical composition containing an eosinophil-inhibitory cytokine in an amount sufficient to inhibit an eosinophil response to a chemoattractant. The cytokine may be MIG and/or IP-10, administered at a dose of about 10 μg/kg to about 10 mg/kg, and the chemoattractant may be eotaxins-1, -2, or -3, MCP-2, -3, -4, or -5, RANTES, and/or MIP-1a. The dose may be systemically administered, for example, intravenously or orally.

Another embodiment of the invention is a palliative method whereby a pharmaceutical composition containing an isolated eosinophil-inhibitory cytokine is administered in an amount to alleviate inflammation in the airway of a patient that is likely or be or that has been exposed to an allergen. The patient may exhibit symptoms of rhinitis, asthma, and/or eczema.

Another embodiment of the invention is a method of inhibiting pulmonary eosinophil recruitment by administering MIG and/or IP-10 in a pharmaceutical composition in an amount to inhibit pulmonary eosinophil recruitment. The patient administered the composition may be asthmatic and/or allergic.

Another embodiment of the invention is a treatment method for an allergic patient by administering a pharmaceutical composition containing at least one cytokine capable of negatively regulating a pulmonary inflammatory cell.

Another embodiment of the invention is a treatment method for an individual with eosinophilia. Either MIG and/or IP-10 is administered at a dose of up to about 10 mg/kg to alter eosinophil migration, tissue recruitment, receptor binding, signal transduction, degranulation, and/or mediator release.

Another embodiment of the invention is a method for alleviating asthma in a patient. MIG is administered in a pharmaceutical composition, thereby inhibiting an interleukin (IL)-13-associated asthmatic response in the patient.

Another embodiment of the invention is a pharmaceutical composition containing MIG and/or IP-10 in a pharmaceutically acceptable formulation and an amount sufficient to alter eosinophil activity in the presence of an allergen. The amount is such that a dose from about 10 μg/kg to about 10 mg/kg can be administered.

Another embodiment of the invention is a pharmaceutical composition containing a cytokine which inhibits at least one eosinophil function in response to an eosinophil-induced stimulus. The cytokine may be MIG and/or IP-10. The stimulus may be an allergen, an allergic reaction, an infection, and/or a chemokine such as eotaxin, IL-13, and/or platelet activating factor. Alternatively, the stimulus may be idiopathic.

Another embodiment of the invention is a pharmaceutical composition containing an isolated Th1-associated chemokine in a pharmaceutically acceptable formulation and in an amount sufficient to inhibit eosinophil activity in the presence of an allergen.

Another embodiment of the invention is a pharmaceutical composition containing a recombinant MIG and/or recombinant IP-10 cytokine in a pharmaceutically acceptable formulation and dose sufficient to inhibit an eosinophil function.

Pretreatment of eosinophils with MIG provided dose-dependent inhibition of eosinophil movement towards the non-CCR3 ligands platelet activating factor (PAF) and leukotriene B₄ (LTB₄) in an epithelial transmigration assay. MIG inhibition was specific for eosinophils; pretreatment of neutrophils with MIG did not inhibit neutrophil movement toward PAF and LTB₄ in the transmigration assay.

Treatment of eosinophils with MIG reduced eotaxin-1 or eotaxin-2-induced actin polymerization. Inhibition of eotaxin-1-induced polymerization was dose dependent. Treatment of eosinophils with MIG reduced actin polymerization in response to the non-CCR3 ligand PAF in a dose-dependent manner.

MIG impaired the subcellar localization of filamentous actin (f-actin). F-actin was present in an altered punctate distribution in MIG-treated eosinophils. In eosinophils treated with both eotaxin-1 and MIG, cortical assembly of f-actin was inhibited. Pretreatment of eosinophils with MIG reduced the amount of eotaxin-1-induced activated Rac-GTPase detected in an immunoprecipitation assay.

Eosinophils isolated from a Rac2 -/- knockout mouse were tested in a transmigration assay for response to eotaxin-2. Transmigration of the Rac2-eosinophils in response to eotaxin-2 was reduced compared to wild-type eosinophils. MIG pretreatment of eosinophils from Rac2 -/- mice had no effect on transmigration in response to eotaxin-2. Rac2 -/- eosinophils already have a reduced transmigration response as compared to wild-type. Thus, Rac2 was required for the effect of MIG on eosinophils. MIG treatment reduced eotoxin-1-induced f-actin assembly in wild-type cells but not Rac2 -/- cells.

MIG had no impact on calcium mobilization in eosinophils. MIG did not inhibit eotaxin-induced calcium mobilization in eosinophils. MIG showed dose-dependent binding to wild-type eosinophils in a flow cytometry assay. MIG did not bind to eosinophils from CCR3 -/- mice. MIG did not inhibit chemotaxis of CCR3 -/- eosinophils towards PAF in a transmigration assay. MIG did not inhibit PAF-induced actin polymerization in CCR3 -/- eosinophils. MIG increased binding of biotinylated eotaxin-1 to wild-type eosinophils in a flow cytometry assay. This suggested that MIG altered CCR3 receptor-ligand interaction.

In summary, the inhibition by MIG of eosinophil functions was more complex than a simple model of receptor antagonism; both CCR3 and non-CCR3 ligands were affected. Rac2 was required for actin polymerization and chemotaxis in eosinophils, and for MIG inhibition of eosinophils. CCR3 was required for MIG inhibition of eosinophils. MIG treatment alone, in the absence of other chemokines, altered actin assembly. MIG inhibited eotaxin-1 induced f-actin assembly. MIG did not affect calcium mobilization. MIG induced conformational changes in CCR3 receptors that induced inhibitory signaling.

These and other embodiments and advantages will be apparent in light of the following figures and detailed description.

### BRIEF DESCRIPTION OF THE DRAWINGS

This application contains at least one drawing executed in color. A Petition under 37 C.F.R. §1.84 requesting acceptance of the color drawings is filed separately on even date herewith.

FIG. 1 demonstrates allergen induction of the cytokines monokine-induced interferon γ (MIG) and IFN-γ-inducible protein of 10 kDa (IP-10). FIGS. 1A and 1B are graphs from microarray hybridization analysis showing induction of monokine-induced interferon γ (MIG) (FIG. 1A) and IP-10 (FIG. 1B) in mice with experimental asthma from control and challenged lung with the ovalbumin allergen. FIG. 1C is a Northern blot of lung ribonucleic acid (RNA) from control and challenged lung at various time points after allergen exposure. FIG. 1D is a Northern blot of lung RNA from control and challenged lung with Aspergillus as the allergen. FIG. 1E is a Northern blot showing the effect of the transcription factor STAT-6 on MIG induction following Aspergillus challenge in wild type and knockout mice. FIG. 1F(A-D) shows the expression pattern of MIG mRNA in ovalbumin-challenged lung by in-situ hybridization.

FIG. 2 shows comparative receptor expression data. FIGS. 2 (A-D) show data from flow cytometry analysis. FIG. 2E is a representative chemotaxis assay showing the effect of MIG on eosinophil migration in vitro.

FIG. 3 shows the inhibitory effect of MIG-pretreatment on eosinophil chemotactic response to eotaxin-2 in vitro.

FIG. 4 shows the effect of MIG-pretreatment on eosinophil migration to lung in vivo. FIG. 4A shows the effect of MIG-pretreatment on eosinophil response to eotaxin-2. FIG. 4B shows the effect of increasing doses of MIG. FIG. 4C shows the effect of eotaxin-1 pretreatment on eosinophil response to eotaxin-2. FIGS. 4D(1-2) show lung tissue with eosinophils detected by anti-MBP immunohistochemistry. FIG. 4E shows the effect of MIG on eotaxin-induced eosinophil mobilization to the blood.

FIG. 5 shows the effect of MIG on eosinophil recruitment to the lung in ovalbumin-induced experimental asthma. FIG. 5A shows the effect of MIG pretreatment on eosinophil recruitment in response to the allergen ovalbumin. FIG. 5B shows the effect of neutralizing MIG prior to ovalbumin challenge using control and anti-MIG antibodies.

FIG. 6 shows the effect on eosinophils of MIG in eosinophils in vitro. FIGS. 6A(1-2) show the specific binding of MIG to eosinophils. FIGS. 6B(1-2) show the lack of internalization of CCR3 by MIG. FIGS. 6C(1-2) show Western blots of eosinophils exposed to eotaxin-2 in the presence and absence of MIG pretreatment, with levels of phosphorylated and total Erk1 and Erk2 shown. FIG. 6D shows the effect of MIG on superoxide production.

FIG. 7 shows the effect of MIG on chemotaxis of eosinophils toward non-CCR3 ligands.

FIG. 8 shows the effect of MIG on leukocyte recruitment to the lung induced by the cytokine IL-13.

FIG. 9 shows MIG inhibition of eosinophil transmigration toward non-CCR3 ligands PAF and LTB₄ in vitro.

FIG. 10 shows MIG inhibition of agonist-induced actin polymerization.

FIG. 11 shows MIG inhibited agonist-induced Rac activation, and the requirement for Rac2 for MIG inhibitory activity.

FIG. 12 shows MIG did not inhibit agonist-induced calcium mobilization.

FIG. 13 shows MIG required CCR3 expression for inhibitory activity.

FIG. 14 shows MIG altered CCR3 receptor ligand interaction.

### DETAILED DESCRIPTION

Chemokines which specifically alter eosinophil function, and methods for their pharmaceutical use, are disclosed. They include monokine induced by interferon-γ (MIG), and an IFN-γ-inducible protein of 10 kDa (IP-10). Their role in therapy for eosinophil-associated diseases and mechanisms of action are also disclosed. As will be appreciated by one skilled in the art, the term cytokine will be used herein to encompass a chemokine.

Chemokines induce signals via seven transmembrane-domain receptors coupled to G proteins, which also form two main subfamilies for CXC and CC chemokines, designated CXCR⁶ and CCR, respectively. Eotaxin (CCL11; now designated eotaxin-1), a CC chemokine with selective activity on eosinophils, has a dominant role in regulating eosinophil baseline homing, and a contributory role in regulating eosinophil tissue recruitment during allergen-induced inflammatory responses. Additional chemokines have been identified in the genome which encode for CC chemokines with eosinophil-selective chemoattractant activity, and have been designated eotaxin-2 (in humans and mice) and eotaxin-3 (in humans only).

The specific activity of eotaxins-1, -2, and -3 is mediated by the selective expression of the eotaxin receptor CCR3 on eosinophils. CCR3 is a promiscuous receptor; it interacts with multiple ligands including macrophage chemoattractant proteins (MCP)-2, -3, and 4, RANTES (regulated upon activation normal T-cell expressed and secreted), and HCC-2 (MIP-5, leukotactin); however, the only ligands that signal exclusively through this receptor are eotaxins-1, -2, and -3, accounting for the cellular selectivity of the eotaxins. CCR3 appears to function as the predominant eosinophil chemokine receptor because CCR3 ligands are generally more potent eosinophil chemoattractants. Furthermore, an inhibitory monoclonal antibody specific for CCR3 blocks the activity of RANTES, a chemokine that could signal through CCR1 or CCR3 in eosinophils. Other cells involved in allergic responses, Th2 cells, basophils, mast cells, and possibly respiratory epithelial cells also express CCR3; however, the significance of CCR3 expression on these cells has been less clearly demonstrated than on eosinophils.

During induction of eosinophil-associated allergic airway inflammation, leukocyte tissue recruitment is orchestrated by the coordinated induction of chemokines. Focusing on eosinophils, a paradigm has emerged implicating Th2 cytokines in the induction of eosinophil active chemokines. For example, IL-4 and IL-13 are potent inducers of eotaxins and MCPs in vitro. When Th2 cytokines are over-expressed or administered to the lung, there is marked induction of eotaxins, as well as strong eosinophil lung recruitment.

In contrast, Th1 cytokines, such as interferon-γ (IFN-γ) induce a different set of chemokines (e.g. an IFN-γ-inducible protein of 10 kDa termed IP-10 or CXCL10; monokine induced by interferon termed MIG or CXCL9; and IFN-inducible T cell α chemoattractant termed I-TAC or CXCL11). These chemokines are unique in that they selectively signal through CXCR3, a receptor expressed on activated T cells (preferentially of the Th1 phenotype), on NK cells, and a significant fraction of circulating CD4 and CD8 T cells. This dichotomy may be even further complex in view of a recent publication indicating that human CXCR3 ligands are human CCR3 antagonists, inhibiting the action of CCR3 ligands on CCR3⁺ cells in vitro (Loetscher et al., J. Biol. Chem. 276:2986 (2001)).

An eosinophil inhibitory pathway mediated by the chemokine CXCL9 (MIG) blocked eosinophil chemoattraction and function in vitro and in vivo. For example, a dosage as low as 1 μg/mouse blocks about 90% of pulmonary eosinophilia induced by eotaxin-2. To our knowledge, this is the first eosinophil inhibitory chemoattraction pathway mediated by a natural protein; accordingly, the existence of this pathway has considerable potential therapeutic significance as the mechanistic pathway may contain a number of targets for drug intervention. The importance of elucidating novel pathways that regulate eosinophil trafficking to the lung has been recently reinforced by the dramatic protection from experimental asthma seen in two different eosinophil-deficient mouse lines.

The mechanism of negative signaling induced by MIG in eosinophils has been determined. In addition to inhibiting eotaxin-induced f-actin formation and chemoattraction, MIG potently blocked PAF- and LTB₄-induced responses, evidencing that MIG's inhibitory activity may be mediated by mechanisms other than CCR3 receptor antagonism alone. MIG-treated eosinophils displayed an abnormal f-actin assembly in the absence of agonist stimulation. However, MIG pre-treatment inhibited eotaxin-induced activation of the Rho GTPase Rac. Rac2 -/- eosinophils displayed a chemotactic and actin assembly defect similar to MIG-treated eosinophils, suggesting Rac2 as a downstream signaling molecule in this inhibitory pathway. Supporting this view, MIG was unable to inhibit eotaxin-induced responses in eosinophils in the absence of Rac2. Using CCR3 gene-targeted cells, MIG's inhibitory activity was demonstrated to be mediated by CCR3. These results evidenced that distinct chemokines can utilize CCR3 to induce opposing signals in eosinophils, with therapeutic ramifications.

Mice overexpressing murine IL-4 under the regulation of the Clara cell 10 promoter (a kind gift of Dr. Jeffrey Whitsett) were used to examine induction of MIG (Rankin et al., Proc. Natl. Acad. Sci. U.S.A. 93:7821 (1996)). All mice were maintained under specific pathogen free conditions and according to institutional guidelines. IL-5 transgenic mice were used as a source of blood and spleen eosinophils.

Asthma was experimentally induced in mice using both ovalbumin (OVA)-induced and Aspergillus fumigatus asthma models. These models are described in Mishra et al., J. Clin. Invest. 107:83 (2001), which is expressly incorporated by reference herein in its entirety. Briefly, for OVA-induced asthma, mice were injected intraperitonally (i.p.) with both OVA and aluminum hydroxide (alum) (1 mg) adjuvant on days 0 and 14, followed by an intranasal OVA or saline challenge on day 24. For Aspergillus-induced asthma, mice received repeated intranasal administrations of Aspergillus fumigatus over the course of three weeks.

Eosinophilia was induced by administration of either eotaxin-2 or IL-13, using procedures described in Rothenberg et al., Molec. Med. 2:334 (1996), which is expressly incorporated by reference herein in its entirety. Mice received 3 μg of recombinant eotaxin-2 (a kind gift of Peprotech, Rocky Hill N.J.), or 4 μg and 10 μg of IL-13, directly into the lung via intratracheal delivery. Briefly, mice were anesthetized with ketamine (5 mg/100 μl) then were positioned upright, after which 20 μl of recombinant eotaxin-2, IL-13, or saline (control) was delivered into the trachea with a pipette (Pipetman®, Gilson, Middleton Wis.).

For delivery of MIG, 200 μl (1 μg) of the recombinant chemokine (Peprotech) was injected into the lateral tail vein 30 minutes prior to the intratracheal or intranasal administration of eotaxin-2 and/or intranasal challenge administration of OVA.

MIG neutralization in OVA-sensitized mice was induced with an intraperitoneal injection of 500 μl (500 μg) of ant-murine MIG (a kind gift of Joshua M. Farber) twenty-four hours prior to a single challenge of OVA or saline. Control groups were injected with an isotope-matched control antibody.

Bronchoalveolar lavage fluid (BALF) and/or lung tissue from allergen-challenged mice was harvested 18 hours after challenge. Mice were euthanized by CO₂ inhalation, a midline neck incision was made, and the trachea was cannulated. The lungs were lavaged twice with 1.0 ml phosphate buffered saline (PBS) containing 1% fetal calf serum (FCS) and 0.5 mM ethylenediaminetetraacetic acid (EDTA). The BALF recovered was centrifuged (400×g for 5 minutes at 4° C.) and resuspended in 200 μl PBS containing 1% FCS and 0.5 mM EDTA. Total cell numbers were counted with a hemocytometer. Cytospin preparations were stained with Giemsa-Diff-Quick (Dade Diagnostics of P.R., Inc., Aguada PR) and differential cell counts were determined. Ribonucleic acid (RNA) from lung was extracted using the Trizol reagent as per the manufacturer's instructions. Following Trizol purification, RNA was repurified with phenol-chloroform extraction and ethanol precipitation.

Microarray hybridization was performed by the Affymetrix Gene Chip Core facility at Children's Hospital Medical Center (Cincinnati Ohio). Briefly, RNA quality was first assessed using the Agilent bioanalyzer (Agilent Technologies, Palo Alto Calif.). Only mRNA having a ratio of 28S/18S between 1.3 and 2 were subsequently used. RNA was converted to cDNA with Superscript choice for cDNA synthesis (Invitrogen, Carlsbad Calif.) and subsequently converted to biotinylated cRNA with Enzo High Yield RNA Transcript labeling kit (Enzo Diagnostics, Farmingdale N.Y.). After hybridization to the murine U74Av2 GeneChip (Affymetrix, Santa Clara Calif.), the chips were automatically washed and stained with streptavidin-phycoerythrin using a fluidics system. The chips were scanned with a Hewlett Packard GeneArray Scanner. The analysis was performed with one mouse per chip (n=3 for each allergen challenge condition, and n=2 for each saline challenge condition).

Levels of gene transcripts were determined from data image files, using algorithms in the Microarray Analysis Suite Version 4 software (Affymetrix). Levels from chip to chip were compared by global scaling; thus, each chip was normalized to an arbitrary value (1500). Each gene is typically represented by a probe set of 16 to 20 probe pairs. Each probe pair consists of a perfect match oligonucleotide and a mismatch oligonucleotide that contains a one base mismatch at a central position. Two measures of gene expression were used, absolute call and average difference. Absolute call is a qualitative measure in which each gene is assigned a call of present, marginal or absent, based on the hybridization of the RNA to the probe set. Average difference is a quantitative measure of the level of gene expression, calculated by taking the difference between mismatch and perfect match of every probe pair and averaging the differences over the entire probe set. Differences between saline and OVA-treated mice were also determined using the GeneSpring software (Silicon Genetics, Redwood City Calif.). Data for each allergen challenge time point were normalized to the average of the saline-treated mice. Gene lists were created with results having p<0.05 and >2-fold change.

Lung tissue samples were fixed with 4% paraformaldehyde in phosphate buffer (pH 7.4), embedded in paraffin, cut into 5 μm sections, and fixed to positive charge slides. For analysis of mucus production, tissue was stained with Periodic Acid Schiff (Poly Scientific R&D Corp.) according to the manufacturer's recommendations. Specifically, a 1.0×1.0 cm grid ocular was used to quantify the percent of epithelial cells that were producing mucus. Five hundred linear gradations (representing 6.25 mm of epithelium) were randomly counted, and the results were expressed as a ratio of mucus producing cells:total pulmonary epithelial cells. For eosinophil staining in tissue, an antiserum against murine major basic protein (anti-MBP) was applied, as described in Matthews et al., Proc. Natl. Acad. Sci. U.S.A. 95:6273 (1998), which is expressly incorporated by reference herein in its entirety. In brief, endogenous peroxidase was quenched with 0.3% hydrogen peroxide in methanol, followed by non-specific protein blocking with normal goat serum. Tissue sections were then incubated with rabbit anti-murine MBP antibody (1:2500, a kind gift from J. Lee, Mayo Clinic, Scottsdale Ariz.) overnight at 4° C., followed by biotinylated goat anti-rabbit IgG secondary antibody (1:200 dilution) and avidin-peroxidase complex (Vector Laboratories) for thirty minutes each. These slides were further treated with nickel diaminobenzidine-cobalt chloride solution to form a black precipitate, and counter-stained with nuclear fast red. Immunoreactive cells were quantitated by morphometric analysis (Metamorph Imaging System, Universal Imaging Corporation, West Chester Pa.) as described in Mishra et al., J. Clin. Invest. 107:83 (2001). The lung sections were taken from the same position in each set of mice and at least 4-5 random sections/mouse were analyzed. Using digital image capture, tissue regions associated with medium sized bronchioles or blood vessels were quantified for the total MBP⁺ cell units relative to the total tissue area. Calculated eosinophil levels were expressed as cells/mm².

Chemotactic responses were determined by transmigration through respiratory epithelial cells as previously described in Zimmermann et al., J. Immunol. 164:1055 (2000), which is expressly incorporated by reference herein in its entirety. In brief, A549 cells (American Type Tissue Culture Collection, Rockville Md.) were grown as monolayers in tissue-culture flasks in Dulbecco's Modified Eagles Medium (DMEM) (Gibco BRL) supplemented with 10% FCS, penicillin, and streptomycin. Cell monolayers were trypsinized, centrifuged, and resuspended in fresh medium prior to culture on permeable filters (polycarbonate filters with 3 μm pores) in Transwell tissue-culture plates (Corning Costar Corp., Cambridge Mass.). Cells (1.5×10⁵) in a volume of 100 μl were grown to confluence on the upper surface of the filters for two days, and treated with 10 ng/ml TNFα for 18 hours. Leukocytes (1.5×10⁶) in Hank's buffered salt solution (HBSS) and 0.5% bovine serum albumin (BSA, low endotoxin, Sigma) were placed in the upper chamber and the chemokine (in HBSS and 0.5% BSA) was placed in the lower chamber. Eosinophils were obtained using splenocytes from IL-5 transgenic mice. Transmigration was allowed to proceed for 1.5 hours. Cells in the lower chamber were counted in a hemocytometer, cytocentrifuged, stained with Giemsa-Diff-quick (Dade Diagnostics of P.R., Inc., Aguada PR), and the differential white cell analysis was determined microscopically.

For flow cytometry analysis, splenocytes (5×10⁵) were washed with FACS-buffer (2% BSA, 0.1% Na-azide in PBS) and incubated for 20 minutes at 4° C. with one of the following: 150 ng (1.5 μg/ml) phycoerythrin-conjugated anti-murine CCR-3 antibody (R&D Systems, Minneapolis Minn.), 300 ng (3 μg/ml) anti-murine CXCR3 (a kind gift of Merck Research Laboratories), 1 μg (10 μg/ml) FITC-conjugated anti-murine CD4 (BD Biosciences Pharmingen, San Diego Calif.), or isotope-matched control IgG. After two washes in FACS-buffer, cells stained for CXCR3 were incubated in the dark with 0.3 μg FITC-conjugated isotope specific secondary antibody (Pharmingen) for 20 minutes at 4° C. After two washes, labeled cells were subjected to flow cytometry on a FACScan flow cytometer (Becton Dickinson) and analyzed using the CELLQuest software (Becton Dickinson). Internalization of surface CCR3 was assayed as described in Zimmermann et al., J. Biol. Chem. 274:12611 (1999), which is expressly incorporated by reference herein in its entirety. Briefly, cells were incubated for 15 minutes at either 4° C. or 37° C., with either 0 ng/ml or 100 ng/ml murine eotaxin-2, or with 1 ng/ml-1000 ng/ml murine MIG. Following chemokine exposure, cells were immediately placed on ice and washed with at least twice the volume of cold FACS buffer.

For MIG binding, cells (5×10⁵) were incubated for 15 minutes at 4° C. with 100 nM to 1000 nM murine MIG. Following chemokine exposure, cells were washed, fixed with 2% paraformaldehyde, washed, and then incubated with 500 ng (5 μg/ml) anti-murine MIG (R&D Systems) at 4° C. for 15 minutes. Cells were stained with phycoerythrin-conjugated secondary antibody. For eotaxin binding, cells (1×10⁶) were exposed to 0 μM to 10 μM of MIG or eotaxin-2 for five minutes at 4° C. prior to incubation with 20 nM biotinylated eotaxin (Sigma) or JE (R&D Systems). Chemokine binding was detected with FITC-conjugated avidin (R&D Systems).

Eosinophils were purified from splenocytes from IL-5 transgenic mice for signal transduction studies following depletion of T and B cells using Dynabeads Mouse pan B (B220) and pan T (Thy 1.2) per the manufacturer's instructions. Purified (85%) eosinophils (1×10⁶) were incubated in RPMI Medium 1640 (Invitrogen Corporation, Carlsbad Calif.) for fifteen minutes at 37° C. prior to stimulation with 10 nM eotaxin-2 and/or 50 nM to 500 nM MIG for two or ten minutes at 37° C. Reactions were stopped with cold PBS with 2 mM sodium orthovanadate (Sigma). Cells were lysed in 25 μl of lysis buffer (5 mM EDTA, 50 mM NaCl, 50 mM NaF, 10 mM Tris-HCl pH 7.6, 1% Triton-X, 0.1% BSA). An equal volume of sample buffer was added to each lysate prior to boiling for five minutes. Samples were separated on a NuPAGE 4-12% Bis-Tris SDS gel (Invitrogen). The proteins were transferred by electroblotting onto nitrocellulose membranes (Invitrogen). The blots were probed with antibodies specific for (1:1000) phospho-p44/42 Map Kinase (Cell Signaling Technologies, Beverly Mass.). Membranes were stripped by incubation at 50° C. for thirty minutes in stripping buffer (100 mM 2-mercaptoethanol, 2% SDS, 62.5 mM Tris-HCl pH 6.7), and then reprobed with antibodies to (1:1000) p44/42 (Cell Signaling Technologies). Proteins were visualized using the ECL system (Amersham Pharmacia Biotech, Piscataway N.J.) after incubating membranes with (1:1500) anti-rabbit IgG HRP (Cell Signaling Technologies).

Data were expressed as mean±standard deviation except where noted. Statistical significance comparing different sets of mice was determined by the Student's t-test.

The following were used for mechanistic determinations.

Eight to twelve week old male and female CD2-IL-5 transgenic mice (BALB/c) were housed under specific pathogen-free conditions and used as a source of eosinophils. The CD2-IL-5 transgenic mice were mated with CCR3 gene targeted BALB/c mice to be used as a source for CCR3-deficient eosinophils. Similarly, Rac2-deficient eosinophils were derived from CD2-IL-5 transgenic Rac2-deficient mice.

Chemotactic responses were determined by transmigration through respiratory epithelial cells as described in Zimmermann et al., Molecular analysis of CCR-3 events in eosinophilic cells, J. Immunol. 2000; 164: 1055-1064, which is expressly incorporated by reference herein in its entirety. Leukocytes (1.5×10⁶) in Hank's buffered salt solution (HBSS)+0.5% bovine serum albumin (BSA, low endotoxin, Sigma) were placed in the upper chamber and the chemoattractant (PAF at 10 nM and LTB₄ at 1 nM in HBSS+0.5% BSA) was placed in the lower chamber. Eosinophils were obtained from splenocytes from IL-5 transgenic mice. In some experiments, eosinophils were purified from the spleen by immunomagnetic negative selection, as described in Rothenberg et al., Murine eotaxin: an eosinophil chemoattractant inducible in endothelial cells and in interleukin 4-induced tumor suppression, Proc Natl Acad Sci USA. 1995; 92: 8960-8964, which is expressly incorporated by reference herein in its entirety. Pretreated cells were incubated with chemokine (MIG or eotaxin-2) for 15 minutes at 37° C., and then washed twice to remove chemokine from the media. Transmigration was allowed to proceed for three hours.

Purified eosinophils (1×10⁶ cells/ml) were loaded with 5 μM Fura-2 AM (Molecular Probes, Eugene Oreg.) in HBSS/1% FBS and incubated at 37° C. for thirty min in the dark. Cells were washed twice with flux buffer (145 mM NaCl, 4 mM KCl, 1 mM NaHPO₄, 0.8 mM MgCl₂, 1.8 mM CaCl₂, 25 mM HEPES, and 22 mM glucose) and resuspended at 1×10⁶ cells/ml. Cells (2 ml) were prewarmed and stimulated in a cuvette with a continuously stirring magnetic bar at 35° C. in a RatioMaster fluorometer (Photon Technology, Inc., South Brunswick N.J.). Data were recorded as the relative ratio of fluorescence emitted at 510 nm after excitation at 340 and 380 nm (y-axis) over time (x-axis).

Agonist-induced actin polymerization was assessed using nitrobenzoxadiazole (NBD)-phallacidin (Molecular Probes). Purified eosinophils were resuspended at 2.5×10⁶ cells/ml in PBS and stimulated with eotaxin (10 nM), PAF (10 nM), MIG (200 nM) or the specified combination at 37° C. for indicated amounts of time. Following stimulation, cells were fixed in 3.7% formaldehyde for sixty minutes. Lysophosphatidylcholine (100 mg/ml, Sigma) and 3.3×10⁻⁷ M NBD-phallacidin were added to the cells and incubated for one hour in the dark. Cells were analyzed on a FACScalibur with a linear fluorescence channel (FL1) where the fluorescence is proportional to f-actin content. Relative f-actin content was expressed as the ratio of the mean channel fluorescence between chemokine and media alone stimulated cells. The percent inhibition of the maximal response was calculated for relative f-actin content for MIG treated cells compared to untreated cells.

To characterize subcellular localization of f-actin, 2.5×10⁴ cells were seeded onto chamber slides (Nalge Nunc International, Naperville Ill.) in RPMI for one-two hours at 37° C. to allow the cells to adhere to the slide. Eosinophils were then stimulated with 10 nM eotaxin-1 for ten seconds, fixed with 2% paraformaldehyde (pH 7.4) (Fisher Scientific) for twenty min and then permeabilized with 0.1% Triton X-100 (Sigma). After incubation in 2% BSA for fifteen min, the cells were stained with rhodamine-labeled phalloidin (Molecular Probes) for one hour at room temperature and then washed three times with PBS. Slides were mounted using SlowFade Light Antifade Kit with DAPI (Molecular Probes). Fluorescence images were acquired on a Leica fluorescence microscope equipped with a deconvolution system (Leica, Germany) driven by Openlab software (Improvision, Lexington Mass.).

GTP bound Rac was precipitated with a PAK-PBD based assay (Upstate Biotechnology, Lake Placid N.Y.). Eosinophils (1×10⁶) in 250 μl of RPMI were stimulated with eotaxin-1 (10 nM) for the indicated times. Pretreated eosinophils were incubated with MIG (200 nM) for two minutes at 37° C. prior to agonist addition. Stimulation was stopped by adding ice-cold PBS. After centrifugation, cell pellets were resuspended in 0.5 ml magnesium-containing lysis buffer (MLB). Aliquots were saved for immunoblot analysis of total Rac. Lysates were rotated with 5 μg PAK-PBD agarose at 4° C. for sixty min. The agarose pellet was washed twice with 1×MLB and resuspended in 30 μl reducing sample buffer. Samples were separated by SDS-PAGE (4-12% Bis-Tris gels, MES-buffer, Invitrogen), transferred to nitrocellulose membranes (Invitrogen) and blotted for pan Rac (BD Transduction Labs, San Diego Calif.).

For MIG binding, wild-type and CCR3-deficient eosinophils (2×10⁵) were incubated for fifteen minutes at 4° C. with 1 nM to 1000 nM murine MIG. Following chemokine exposure, cells were washed, fixed with 2% paraformaldehyde, washed, and then incubated with 500 ng (5 μg/ml) polyclonal goat anti-murine MIG (R&D Systems) or control goat IgG at 4° C. for fifteen minutes. Cells were stained with FITC-conjugated secondary antibody. For eotaxin-1 binding studies, eosinophils were incubated with a competitor chemokine (e.g. MIG at 20 nM, 200 nM, 500 nM or 2000 nM) for five minutes on ice, prior to adding biotinylated 20 nM eotaxin-1 (Sigma) or JE (R & D Systems) for thirty minutes on ice. Cells were stained with streptavidin-PE (BD Pharmingen) for thirty minutes on ice. Chemokine binding was reported as % mean channel fluorescence over baseline (no chemokine or no competitor) mean channel fluorescence.

Identification of CXCR3 Ligands in Experimental Asthma

Genes differentially expressed in a well established model of asthma were identified. To induce asthma, mice were intraperitonally sensitized with the allergen OVA in the presence of the adjuvant alum on two separate occasions separated by 14 days. Subsequently, replicate mice were challenged with intranasal OVA or control saline on two occasions separated by 3 days. Three and/or eighteen hours after each allergen challenge, lung RNA was subjected to microarray analysis utilizing the Affymetrix chip U74Av2 that contained oligonucleotide probe sets representing 12,423 genetic elements, the largest collection of characterized mouse genes commercially available.

Comparison of allergen-challenged mice to saline-challenged mice revealed a>2-fold change in 2-6% of the genes, at the various time points (data not shown). Of these allergen-induced genes, the genes encoding chemokines represented a large subset. For example, the microarray chip contained oligonucleotides that represented 29 chemokine genes; 10 of these were allergen-induced, compared with saline challenged control mice. Several of the induced chemokine genes were not previously associated with allergic lung responses. For example, there was strong induction of the IFN-γ inducible chemokines MIG and IP-10.

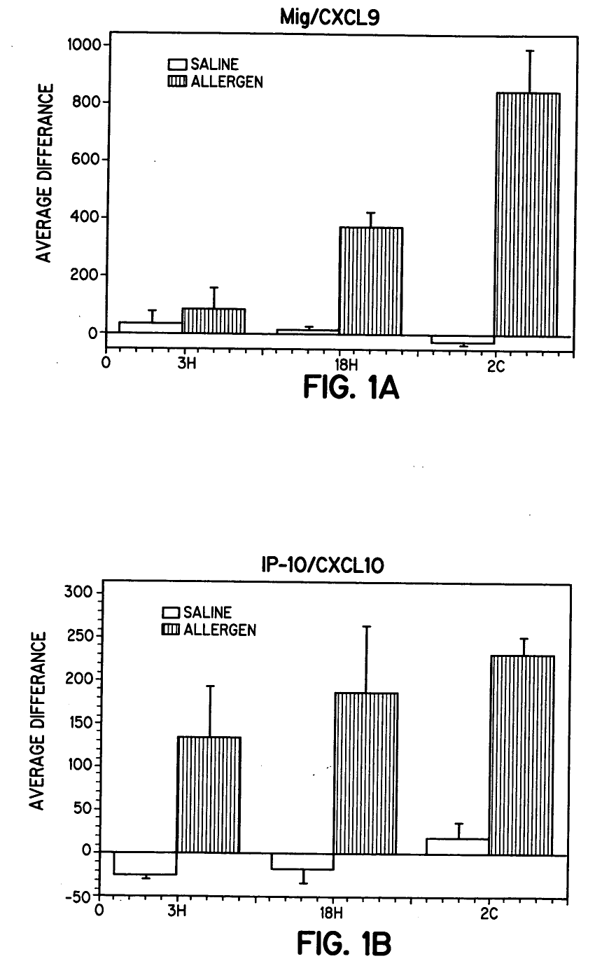

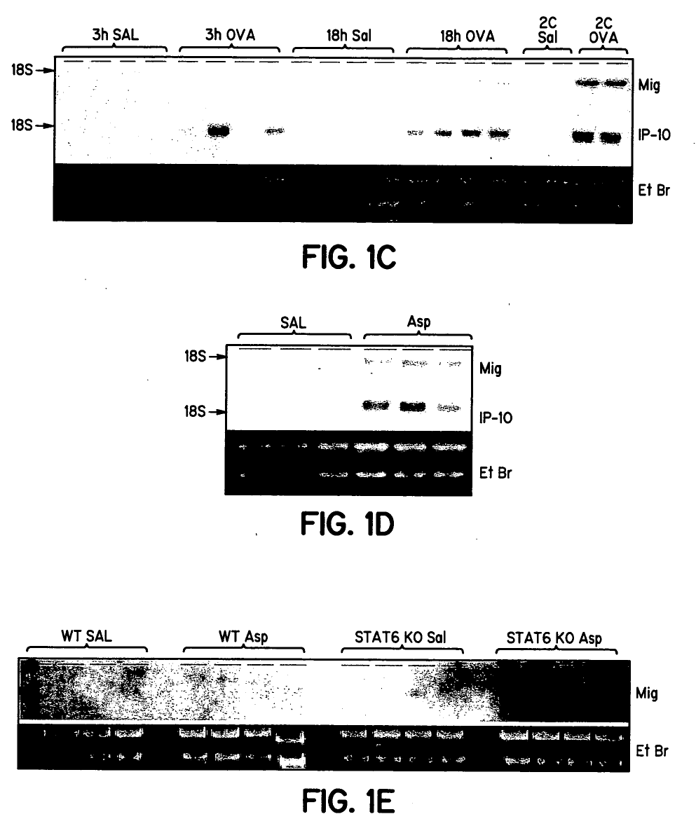

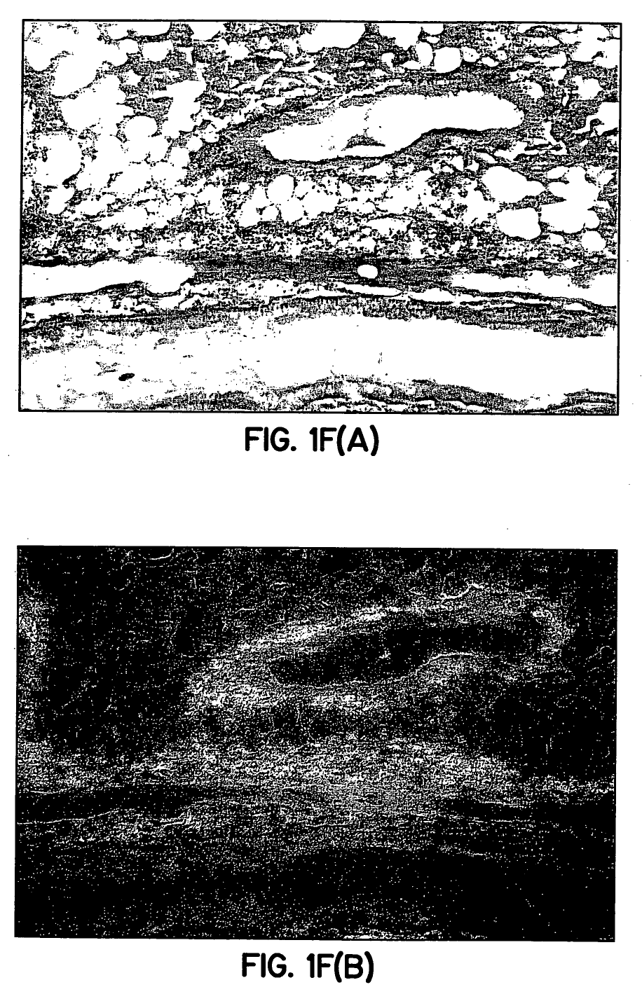

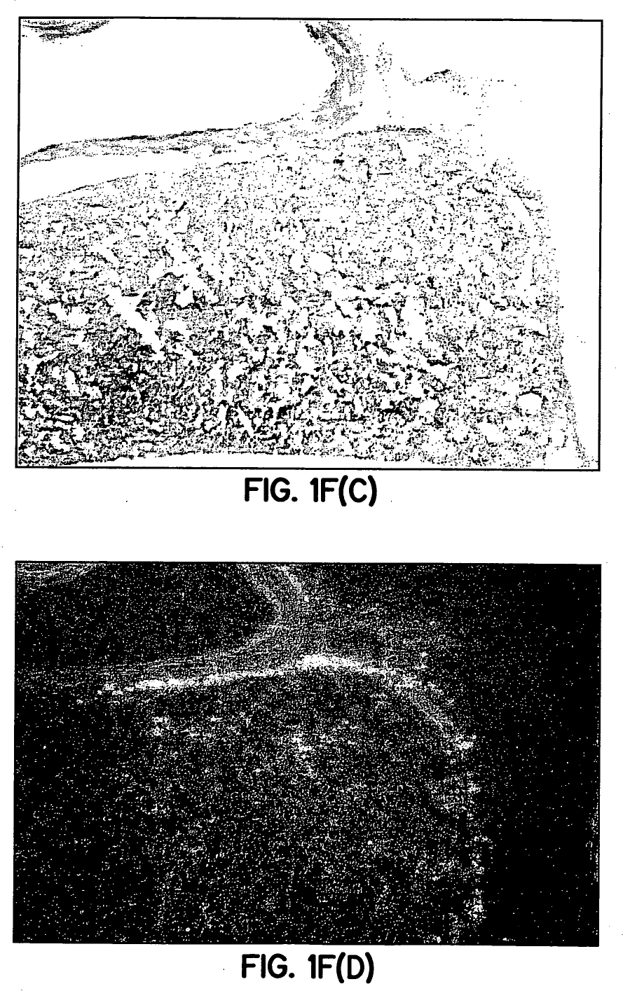

FIG. 1 shows MIG and IP-10 mRNA expression in ovalbumin (OVA) or Aspergillus fumigatus-induced asthma models. 1A shows the average difference (mean and standard error of the mean) for the hybridization signal of MIG in saline (control) and OVA challenged mice. 1B shows results for IP-10 at three (3 H) and eighteen hours (18 H) following one challenge, and eighteen hours following two challenges (2 C). 1C shows MIG and IP-10 mRNA expression in saline and OVA challenged mice. FIG. 1D shows MIG and IP-10 mRNA expression following saline or Aspergillus challenge. FIG. 1E shows MIG mRNA expression in wild-type and STAT-6 deficient mice following saline or Aspergillus challenge. The location of 18S RNA is shown; the RNA gels were stained with ethidium bromide. Each lane represents RNA from a single mouse.

MIG mRNA was increased by >10-fold 18 h after the first allergen challenge (FIG. 1A, 18 H), and >>10-fold after the second allergen challenge (FIG. 1A, 2 C), compared to saline controls. IP-10 mRNA was increased by >10-fold 18 h after the first allergen challenge (FIG. 1B, 18 H), and >10-fold after the second allergen challenge (FIG. 1B, 2 C) compared to saline controls.

To verify that the microarray data reflected gene induction, Northern blot analysis was performed. As shown in FIG. 1C, MIG and IP-10 mRNA were strongly inducible following allergen challenge. Bands for MIG mRNA appeared at 18 h after the first challenge, and were more intense after the second challenge. Bands for IP-10 mRNA appeared 3 h after the first challenge, as well as 18 h after the first challenge and after the second challenge. Thus, the Northern blot analysis substantiated the microarray data.

To determine if the induction of MIG and IP-10 was limited to the OVA-induced model of experimental asthma, experimental asthma was induced in naive mice by repeated intranasal doses of the antigen Aspergillus fumigatus. Eighteen hours after the last of nine doses of intranasally administered Aspergillus fumigatus, lung RNA was subjected to Northern blot analysis and probed for MIG and IP-10. The results are shown in FIG. 1D.

Compared with mice challenged with nine doses of intranasal saline, mice challenged with Aspergillus fumigatus had marked expression of MIG and IP-10. Thus, the induction of MIG and IP-10 by allergen challenge was not specific to the antigen employed.

Regulation of MIG Expression

The effect of cytokines that are known to be overexpressed in the asthmatic lung, for example IL-4 and IL-13, on the induction of MIG expression, were determined. Specifically, MIG expression in transgenic mice that over-express IL-4 was determined; IL-4 overexpression did not induce MIG expression (data not shown). MIG expression in mice administered IL-13 to the lungs via the intranasal route was also determined; IL-13 administration did not induce MIG expression (data not shown). In contrast, under the same condition, IL-4 and IL-13 induced eotaxin-1 and eotaxin-2 expression.

IL-4 and IL-13 share a common receptor signaling pathway involving post-receptor events that are usually dependent on the transcription factor STAT-6. Thus, the effect of the protein STAT-6 on MIG induction was determined by inducing experimental asthma in both STAT-6 wild type mice and STAT-6 gene-deleted mice. The results are shown in FIG. 1E. Compared to STAT-6 wild-type mice, allergen-induced MIG was enhanced in STAT-6 knockout mice. These results contrasted with the induction of eotaxin-1 and eotaxin-2, which were completely dependant upon STAT-6 (data not shown).

FIG. 1F shows MIG mRNA expression in allergen-challenged lung. There was predominant peri-vascular and peri-bronchial expression of MIG RNA in OVA-challenged lung. This was seen by bright field (FIG. 1F(a)) and dark field (FIG. 1F(b)) in situ hybridization of MIG mRNA (10× magnification). FIGS. 1F(c) and 1F(d) show expression of MIG in lung lymph node from OVA-challenged lung in bright field and dark field in situ hybridization, respectively (40× magnification).

Murine MIG is an Inhibitor of Eosinophils In Vitro

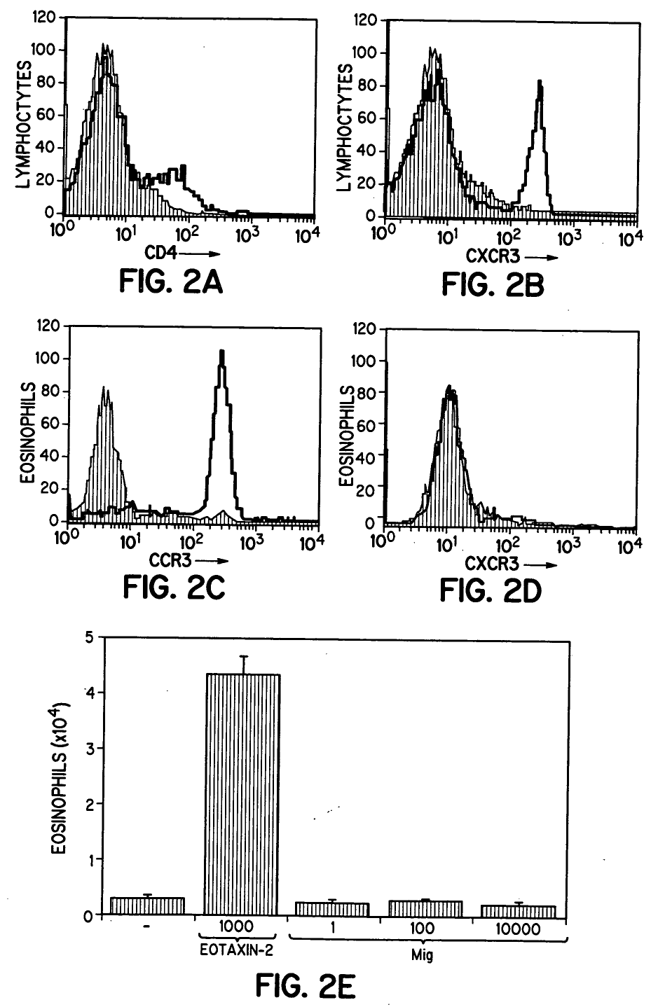

Human eosinophils have been reported to express CXCR3, which is the receptor for MIG. To determine if allergen-induced expression of MIG could be responsible, at least in part, for recruitment of eosinophils to the lung, it was first determined whether murine eosinophils expressed CXCR3. The results showing failure of murine eosinophils, which lack CXCR3 on their surface, to migrate toward MIG are shown in FIG. 2. FIG. 2A shows lymphocytes from IL-5 transgenic mice which express CXCR3; eosinophils have no detectable CXCR3 on their surface. The filled histogram is the isotope-matched control, and the solid line is CCR3, CXCR3, or CD4. FIG. 2B shows a representative result (n=3) of transmigration of spleen-derived eosinophils in response to doses of MIG as indicated. Cells (1.5×10⁶) were allowed to transmigrate in response to MIG and eotaxin-2. Cells were counted in the lower chamber 1.5 hours later. Data represent mean and standard deviation of eosinophils that migrated through a layer of respiratory epithelial cells.

The histograms in FIG. 2A show flow cytometry data from eosinophils and lymphocytes. CD4+ lymphocytes expressed the CXCR3 receptor on the cell surface. In contrast, eosinophils, identified by their characteristic light scatter and expression of the eotaxin receptor CCR3, had no detectable expression of the MIG receptor CXCR3. In contrast to the strong expression of CCR3 in eosinophils, there was no reproducible staining for CXCR3 on eosinophils (n=3 experiments) using a range of antibody doses (0.5 μg/ml to 30 μg/ml). As a control, CXCR3 was identified in CD4+ T cells.

It was next determined if MIG could induce eosinophil migration in vitro. FIG. 2B shows a representative transmigration assay for lung eosinophils. Consistent with the absence of CXCR3 expression, murine eosinophils did not respond by transmigration when subjected to a range of doses of MIG (1 pg/ml to 10,000 pg/ml). As a positive control, eosinophils strongly responded to 1000 pg/ml of eotaxin-2. These data suggest that MIG was not a stimulatory chemokine for murine eosinophils.

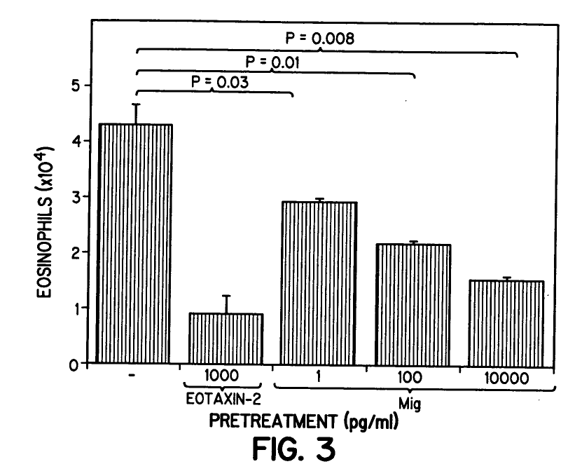

MIG was, however, a functional inhibitor for CCR3 ligand-induced eosinophil chemoattraction in vitro. Eosinophils were pretreated with MIG, and their subsequent chemotactic response to the potent CCR3 ligand, eotaxin-2, was evaluated. The results are shown in FIG. 3.

FIG. 3 shows data for eosinophil transmigration in response to eotaxin-2; MIG inhibited eosinophil migration toward eotaxin-2 in vitro. Cells were allowed to transmigrate following pretreatment with buffer, MIG, or eotaxin-2. Data (mean and standard deviation) are from a representative experiment (n=3) of eosinophils that migrated toward 1 ng/ml eotaxin-2.

Pretreatment of the eosinophils with MIG inhibited eosinophil transmigration in response to eotaxin-2. This effect was seen in a dose-dependent manner, with inhibition of activity noted between 1 pg/ml to 10,000 pg/ml (0.8 pM to 820 pM); p=0.03 at 1 pg/ml, p=0.01 at 100 pg/ml, and p=0.008 at 10,000 pg/ml MIG. As a positive control, pretreatment of eosinophils with eotaxin-2 at a dose of 1 ng/ml (0.1 nM) also inhibited eosinophil transmigration. As a negative control, pretreatment of eosinophils with MCP-1 (JE, CCL2) at a dose of 10 ng/ml (0.7 nM) did not inhibit eosinophil transmigration (data not shown). MIG was not toxic to eosinophils, as determined by exclusion of a viability dye (Trypan blue), and by the ability of IL-5 to promote eosinophil survival even in the presence of MIG (data not shown).

Effect of MIG on CCR3 Internalization

CCR3 ligands induce receptor internalization following receptor engagement with agonists. MIG-induced CCR3 internalization could account for the ability of MIG to inhibit the transmigration of eosinophils that are induced by eotaxin.

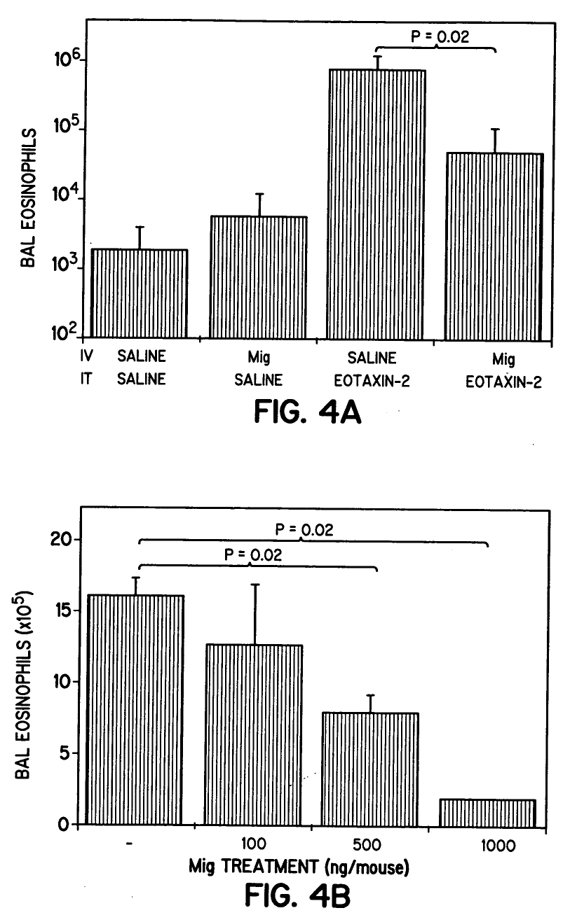

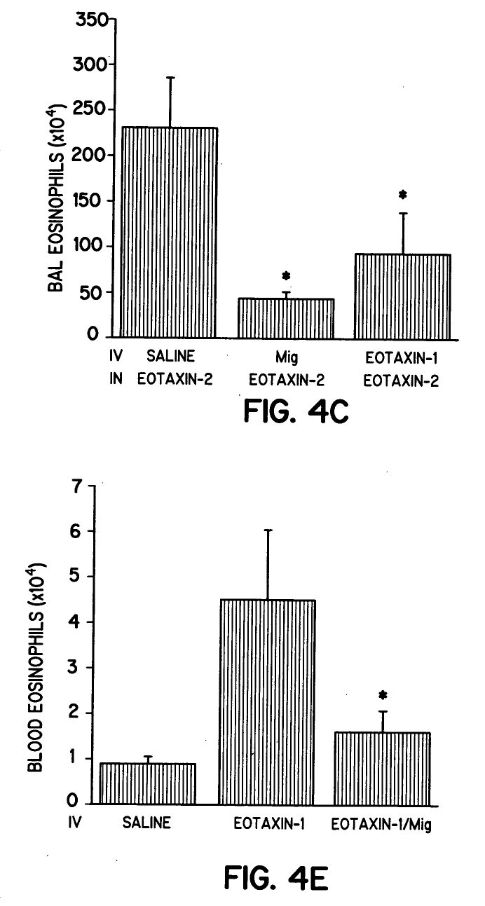

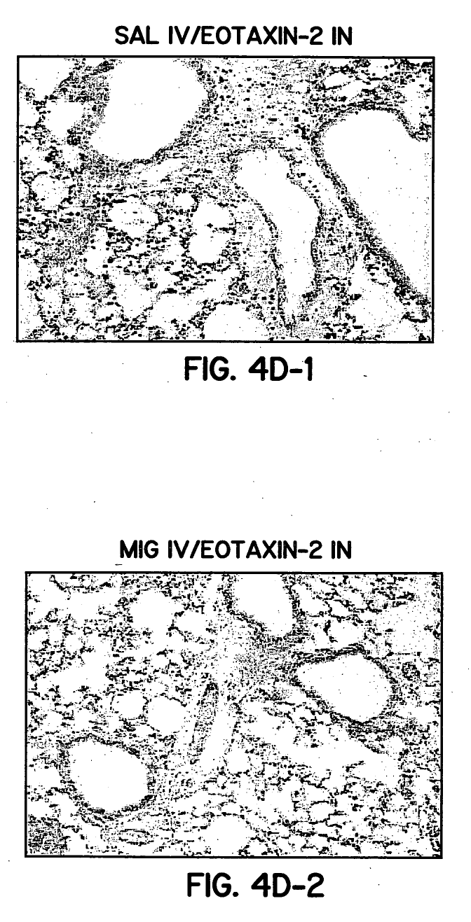

FIG. 4 shows dose-dependent MIG inhibition of chemokine-induced eosinophil recruitment to the lung. FIG. 4A shows the mean and standard deviation of eosinophils that migrated into the airway towards eotaxin-2. IL-5 transgenic mice were treated intravenously with saline or 1 μg MIG thirty minutes prior to intratracheal challenge with 3 μg eotaxin-2 or saline. Data represent three independent experiments, with two to six mice in each group. FIG. 4B shows mice treated with saline or MIG at the doses indicated prior to challenge with eotaxin-2. Data (mean and standard deviation) show airway eosinophils from a representative experiment (n=2), with four mice in each group per experiment.

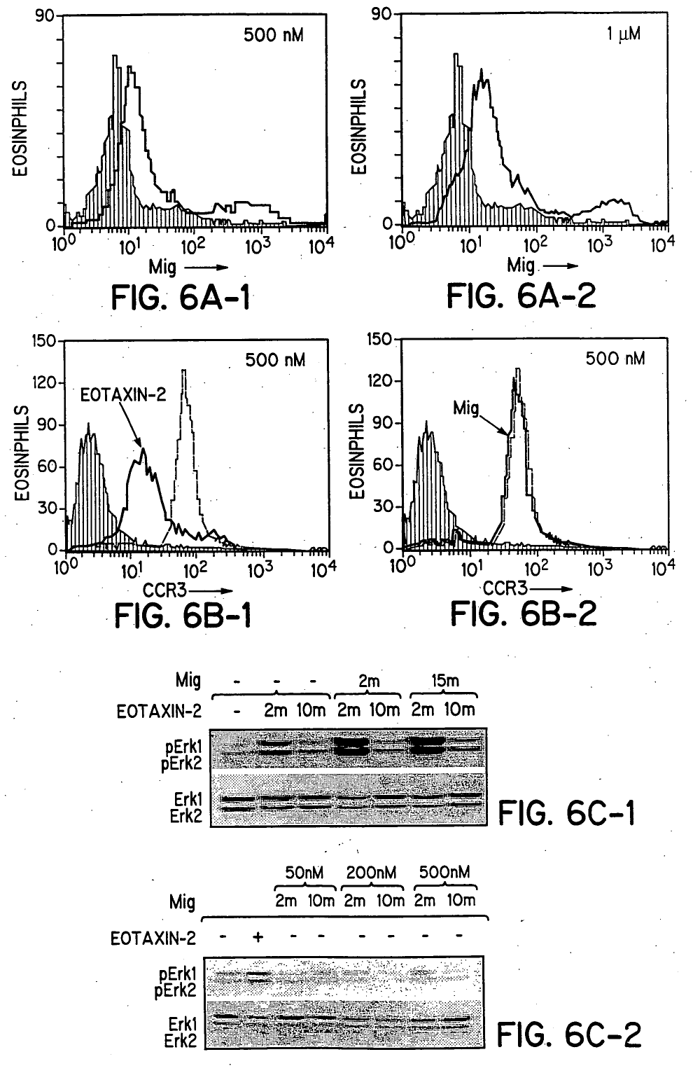

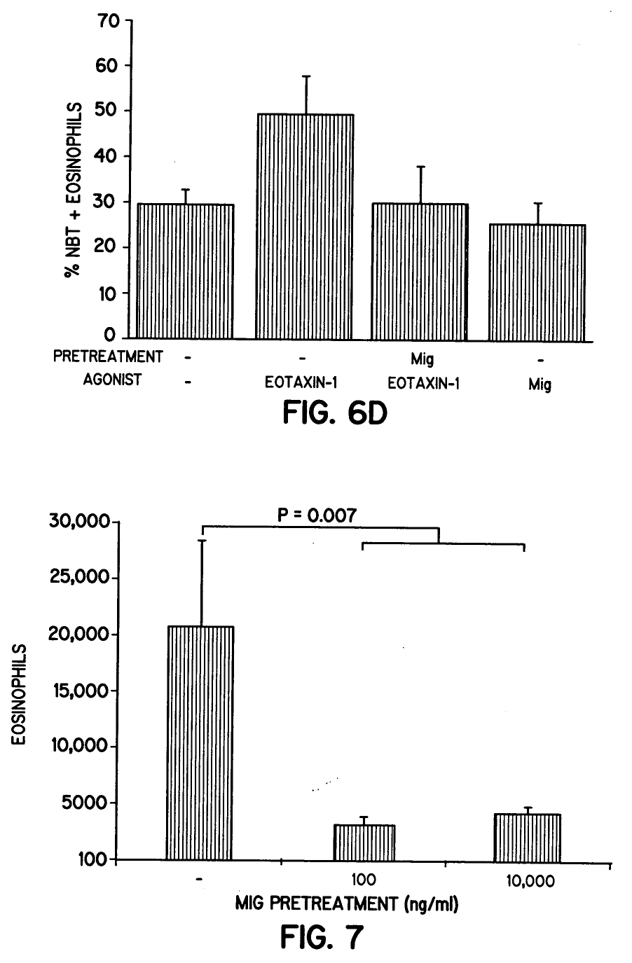

As shown in FIG. 6B, pretreatment of eosinophils with MIG at doses of 1 ng/ml, 100 ng/ml, or 1000 ng/ml (82 pM to 82 nM) did not significantly affect the level of CCR3. As a control, pretreatment of eosinophils with 100 ng/ml (9.7 nM) eotaxin-2 induced marked internalization of CCR3 expression. The effect of eotaxin-2 was not seen when the pre-incubation was conducted at 4° C., verifying that the assay was detecting receptor internalization rather than epitope blockade.

MIG Inhibits Eosinophil Recruitment to the Lung Induced by Eotaxin and IL-13

The effect of MIG to serve as an eosinophil inhibitor in vivo was determined. The ability of MIG to inhibit eosinophil recruitment into the lung, induced by either eotaxin-2 or IL-13, was evaluated. Pretreatment of mice with 1, μg MIG administered intravenously thirty minutes prior to eotaxin-2 administered intratracheally resulted in >90% inhibition in eosinophil trafficking to the lung (FIG. 4A). MIG demonstrated dose-dependent inhibition between 0.1 μg MIG to 1.0 μg MIG (FIG. 4B).

Eotaxin-2, administered intratracheally to IL-5 transgenic mice, induced marked recruitment of eosinophils into the lung. For example, and with reference to FIG. 4A, eosinophil levels in BALF three hours after eotaxin-2 treatment increased from 1.9±2.3×10³ (n=2 mice) to 7.2±5.4×10⁵ (n=6 mice). However, intravenous injection of mice with MIG (1 μg) thirty minutes prior to intratracheal eotaxin-2 administration reduced recruitment of eosinophils, compared to intravenous injection of saline (p<0.02).

Intravenous administration of MIG thirty minutes prior to intranasal administration of eotaxin-2 inhibited eosinophil recruitment into the lung in a dose-dependent manner. With reference to FIG. 4B, intravenous administration of MIG to IL-5 transgenic mice at a dose of 100 ng reduced eosinophil recruitment into BALF by 21%. Intravenous administration of MIG at a dose of 500 ng reduced eosinophil recruitment into BALF by 51% (p=0.02). Intravenous administration of MIG at a dose of 1000 ng reduced eosinophil recruitment into BALF by 88% (p=0.01). To verify that MIG was specifically responsible for the inhibitory activity, mice were treated with a different chemokine, murine MCP-1 (also known as JE) (1 μg), prior to intranasal administration of eotaxin-2. The chemokine JE had no effect on eotaxin-induced eosinophil recruitment in the lung (data not shown). For comparison, mice were intravenously administered 1 μg of eotaxin-1 prior to intranasal eotaxin-2 administration, and eosinophil migration into lung in response was determined. As shown in FIG. 4C, MIG and eotaxin-1 had comparable inhibitory activity. Data represent mean±standard of deviation of lung or airway eosinophils in a representative experiment (n=2) with four mice in each group per experiment. (*p 0.04).

Eosinophil levels in lung tissue were assessed by histological examination and by anti-MBP staining. Eosinophil migration was inhibited after intravenous MIG treatment prior to eotaxin-2 intranasal delivery. The results are shown in FIG. 4D with eosinophils detected by anti-MBP immunohistochemistry in lung following intravenous treatment of saline or MIG prior to intranasal administration of eotaxin-2. MIG (1 μg) administered intranasally prior to eotaxin-2 administration did not significantly inhibit eosinophil recruitment (data not shown). Thus, MIG activity appeared to depend on systemic, rather than local, administration. In contrast to the inhibitory effect of MIG on eosinophil recruitment into either BALF or lung tissue, eosinophil levels in the blood were not affected by MIG at any of the doses administered.

The ability of MIG to inhibit eosinophil chemokine responses in vivo was not limited to eotaxin-2; MIG also inhibited the effects of eotaxin-1. Pretreatment with 1 μg MIG reduced eotaxin-1 induced BALF eosinophilia from 2.6±0.42×10⁶ to 4.0±1.6×10⁵ cells (n=3 mice/group). MIG also inhibited eotaxin-induced eosinophil mobilization to the blood. After intravenous administration of 1 μg eotaxin-1, eotaxin-1 induced a rapid increase in circulating eosinophil levels. The results are shown in FIG. 4E. When 1 μg MIG was administered in combination with eotaxin-1, eotaxin-induced eosinophil mobilization was significantly reduced (*p<0.0001) (3 experiments with 12 mice in each group).

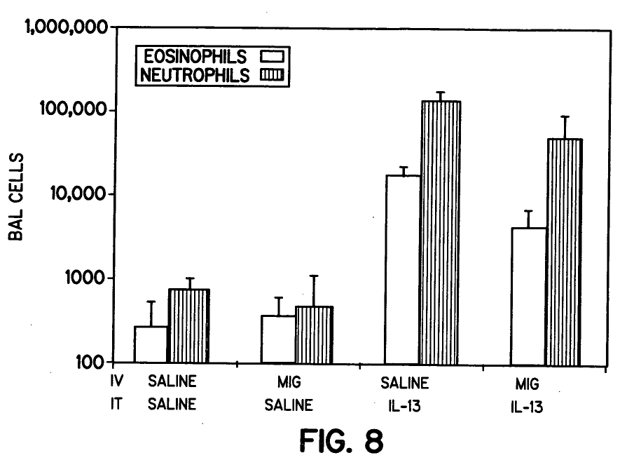

MIG inhibited IL-13-induced granulocyte trafficking to the lung in vivo. IL-13 treated mice were treated intravenously with saline or MIG thirty minutes prior to a second dose of IL-13. The results are shown in FIG. 8; data represent the mean and standard deviation of eosinophils and neutrophils in BALF following treatment, and show a representative experiment (n=2).

IL-13, administered intratracheally to naive Balb/c mice, also induced marked recruitment of eosinophils into the lung. IL-13 was administered at a dose of 4 μg. After two days, the mice were administered an intravenous injection of MIG (1 μg), followed by a second dose of IL-13 (10 μg), again administered intratracheally. After three days, the cell content of the BALF was examined.

With reference to FIG. 8, BALF from IL-13 dosed mice pretreated with MIG (MIG IV/IL-13 IT) had decreased eosinophils (4.2±2.7×10³ (n=4 mice) compared to mice pretreated with saline (control, Sal IV/IL-13 IT) (17.8±5.0×10³ (n=4 mice) (p=0.003). BALF from mice treated with IL-13 following treatment with MIG also had decreased neutrophils (5.1±3.7×10⁴ (n=4 mice) compared to mice pretreated with saline (control, Sal IV/IL-13 IT) (13.2±3.6×10 (n=4 mice) (p=0.02). These data indicate that intravenous MIG inhibited eosinophil recruitment into the lung in response to diverse stimuli. The finding that neutrophil levels in the lung were also inhibited by MIG suggests its generalized ability to block leukocyte trafficking, and more particularly granulocyte trafficking, to the lung. The ability of MIG to block the action of IL-13 is beneficial from a therapeutic vantage, because IL-13 is considered to be a central and critical cytokine in the pathogenesis of asthma.

MIG Inhibits OVA-Induced Eosinophil Recruitment to the Lung

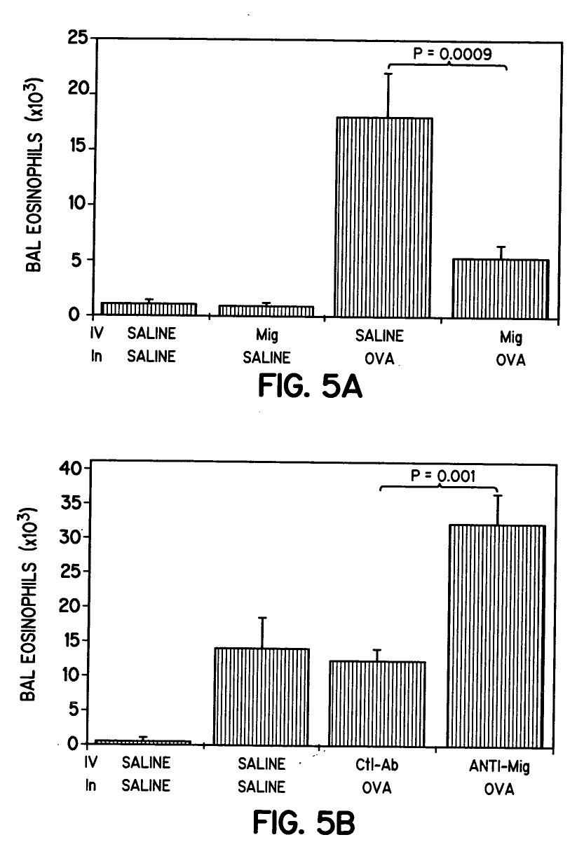

To determine if pharmacological administration of MIG down-regulated eosinophil recruitment to the lung in the OVA-induced experimental asthma model, mice sensitized with OVA were subjected to one challenge with intranasal OVA or saline. The ability of MIG, administered intravenously 30 minutes prior to allergen challenge, to inhibit leukocyte recruitment into the lung was determined. FIG. 5 shows that MIG inhibited allergen-induced eosinophil recruitment to the lung and functioned as an eosinophil inhibitor in vivo. FIG. 5A shows a representative experiment (n=2), with four mice in each group per experiment, of OVA-challenged mice treated with intravenous saline or MIG (1 μg) thirty minutes prior to intranasal OVA challenge. FIG. 5B shows MIG neutralization increased antigen-induced eosinophil recruitment to the lung. OVA-challenged mice were treated with an intraperitoneal injection of 500 μg anti-MIG antibody or isotope-matched control antibody (Ctl-Ab). The results are from a representative experiment (n=2) with two to four mice in each group per experiment. Data from FIGS. 5A and 5B represent the mean and standard deviation of airway eosinophils.

Control mice (saline injection) challenged with OVA demonstrated an increased total leukocyte count in BALF. Eosinophils in BALF increased from 9.1±4.0×10² (IV Saline, IN Saline) to 1.7±0.4×10⁴ (IV Saline, IN OVA)(p=0.08). Total leukocytes in BALF increased from 5.3±0.7×10⁴ (n=3) to 10.2±2.1×10⁴ (n=4). Neutrophils increased in BALF increased from 8.1±4.4×10³ to 4.7±1.4×10⁴ (p=0.005).

Mice administered MIG thirty minutes prior to challenge with OVA had decreased eosinophils in BALF. In comparison to mice receiving saline prior to OVA challenge (IV Saline, IN OVA), mice receiving MIG prior to OVA challenge (IV MIG, IN OVA) demonstrated a 70% reduction (p=0.0009) in eosinophils in BALF. This reduction was specific to eosinophils; mice receiving MIG prior to OVA challenge had no reduction in either BALF neutrophils or lymphocytes (data not shown). This reduction was also specific to MIG; mice receiving the cytokine JE (1 μg) prior to OVA challenge showed no change compared to mice receiving saline prior to OVA challenge (data not shown).

The ability of endogenously expressed MIG, in contrast to pharmaceutically administered MIG to inhibit eosinophil migration in vivo was assessed. It was expected that OVA-challenged mice with decreased or absent MIG would demonstrate increased eosinophil recruitment into the BALF. Mice sensitized with OVA were administered anti-murine MIG (500 μg) twenty-four hours prior to one intranasal challenge with OVA. Eosinophil recruitment into the BALF was then evaluated. The results are shown in FIG. 5B.

IgG control treated mice, challenged with OVA, increased eosinophil recruitment into the airway. Eosinophils in BALF in control mice were 5.0±4.5×10² (IP Saline, IN Saline), while eosinophils in BALF in mice receiving a control antibody (IP CTL-Ab, IN OVA) were 1.2±0.2×10⁴ Treatment of mice with anti-MIG antibody increased eosinophils in BALF greater than two-fold over isotope control treated mice. Eosinophils in BALF in isotope control treated mice were 1.2±0.2×10⁴, while eosinophils in BALF in anti-MIG treated mice were 3.3±0.4×10⁴, following OVA challenge.

Direct Binding of MIG on Murine Eosinophils

The nature of the effect of MIG on eosinophils was determined by evaluating whether MIG bound to eosinophils in vitro. Eosinophil preparations were prepared from the spleen of IL-5 transgenic mice; those mice have large numbers of eosinophils in the spleen and serve as a convenient source of murine eosinophils. Splenocytes were exposed to MIG at one of four doses (100 nM to 1 μM). The binding of MIG to splenocyte eosinophils was evaluated by flow cytometry using anti-MIG antiserum. MIG bound to the surface of eosinophils and attenuated eotaxin-2 signal transduction, as shown in FIG. 6A. MIG bound in a dose-dependent manner to the surface of murine eosinophils from IL-5 transgenic mice. In comparison, the filled histogram represents no binding when no chemokine is present. FIG. 6B shows that MIG did not induce CCR3 internalization. Analysis of surface CCR3 on eosinophils following incubation with buffer (solid line), eotaxin-2 (dotted line), or MIG (dashed line). The filled histogram shows results from isotope matched controls. FIG. 6C shows enhanced eotaxin-2 induced phosphorylation of p44/42 (Erk 1 and Erk 2) in eosinophils following MIG pretreatment. Cells were incubated with buffer, MIG, and/or eotaxin-2 at the indicated time and dose. Phosphorylation of p44/42 was determined by Western blot analysis. The results are from a representative experiment (n=2).

Increased doses of MIG resulted in increased binding of MIG to the surface of eosinophils, compared to eosinophils that were not exposed to MIG or another cytokine. Exposure of eosinophils to 500 nM and 1 μM MIG showed a dose-dependent increase in MIG binding. As a negative control, eosinophils were exposed to the cytokine JE, a ligand of CCR2 which is not normally expressed by murine eosinophils. Binding of MIG to eosinophils was greater than binding of JE to eosinophils (data not shown), even in the absence of detectable expression of CXCR3 on the surface of murine eosinophils.

To determine if CCR3 was the MIG receptor in eosinophils, the ability of MIG to compete for the binding of biotinylated eotaxin-1 to eosinophils was determined. While unlabeled eotaxin-1 or eotaxin-2 was able to compete for biotinylated eotaxin-1 binding to eosinophils, unlabeled MIG at doses up to 10 μm did not inhibit the binding CCR3 ligands (data not shown). Because MIG interaction with CCR3 should result in decreased binding of MIG to eosinophils with internalization of CCR3, the effect of MIG binding was determined with eosinophils that had been pretreated with eotaxin-2 under conditions that promoted marked CCR3 internalization. Under these conditions, MIG binding was not inhibited (data not shown). Therefore, in contrast to the proposal set forth by Loetscher et al. (J. Biol. Chem. 276:2986 (2001), the ability of MIG to inhibit eosinophil responses was not simply related to competitive antagonism of CCR3.

Activation of receptors by chemokines leads to a cascade of intracellular signaling and multiple phosphorylation events. Mitogen activated protein (MAP) kinases are phosphorylated and activated after exposing human eosinophils to CCR3 ligands. The effect of MIG on CCR3-ligand induced signal transduction was therefore determined. Specifically, the phosphorylation activity of two MAP kinases (p44 MAP kinase phosphorylating Erk1, and p42 MAP kinase phosphorylating Erk2) was assayed in eosinophils exposed to eotaxin-2 in the presence and absence of MIG pretreatment. The results are shown as Western blots in FIG. 6C.

In control eosinophils (exposed to eotaxin-2 without MIG pretreatment), phosphorylation by both p44 and p42 was maximal at two minutes, and then was down-regulated at ten minutes after eotaxin-2 exposure. No phosphorylation by either p44 or p42 was detected with only MIG exposure (no eotaxin-2) at any MIG dose (50 nM to 100 nM). FIG. 6C shows the effect of pretreatment with MIG at 50 nM. Eosinophils pretreated with MIG, followed by eotaxin-2 exposure, demonstrated increased Erk1 and Erk2 phosphorylation compared with eosinophils exposed to eotaxin-2 above.

MIG Inhibits Functional Response in Eosinophils

Eosinophils were pretreated with MIG and then treated with eotaxin-1 (10 nM). Eotaxin activation of eosinophils increased nitrobluetetrazolium positive (NBT⁺) cells, indicating superoxide anion and related reactive oxygen species. As shown in FIG. 6D, MIG (100 nM) pretreatment inhibited eotaxin-induced NBT⁺ eosinophils by 94%. Results represent percentage of positive cells, with error bars showing mean±standard deviation, n=3.

MIG Inhibits Eosinophil Responses to Diverse Chemoattractants.

The inhibition of MIG on allergen-induced eosinophilia in the lung was surprising in view of the recent finding that CCR3 deficient mice exhibited about a 50% reduction in BALF eosinophilia following sensitization and challenge with OVA. Therefore, in addition to inhibiting CCR3-mediated pathways in eosinophils, MIG could also inhibit chemoattractants that signal through additional pathways. This was consistent with results that MIG also blocked IL-13-induced eosinophil lung recruitment, because IL-13 induces multiple eosinophil chemoattractants.

The ability of MIG to alter chemotaxis of eosinophils toward a non-CCR3 ligand was evaluated. Eosinophils were pretreated with MIG at a dose of either 100 ng/ml or 10000 ng/ml. Their subsequent chemotactic response to platelet activating factor (PAF) (1 μm) was evaluated. FIG. 7 shows that MIG inhibited migration of eosinophils toward PAF in vivo. Cells were allowed to transmigrate toward PAF following pretreatment with buffer or MIG. The data (mean and standard deviation) are from a representative experiment (n=2) and show eosinophils that migrated toward 1 μm PAF.

As shown in FIG. 7, MIG pre-treatment inhibited eosinophil migration in response to PAF (p=0.007). Thus, MIG induced inhibition was not limited to CCR-3 ligands, and altered eosinophil responses to diverse chemoattractants. These data indicated that MIG induced functional non-responsiveness of eosinophils. While not being bound by a specific theory, such non-responsiveness may be due to heterologous receptor desensitization.

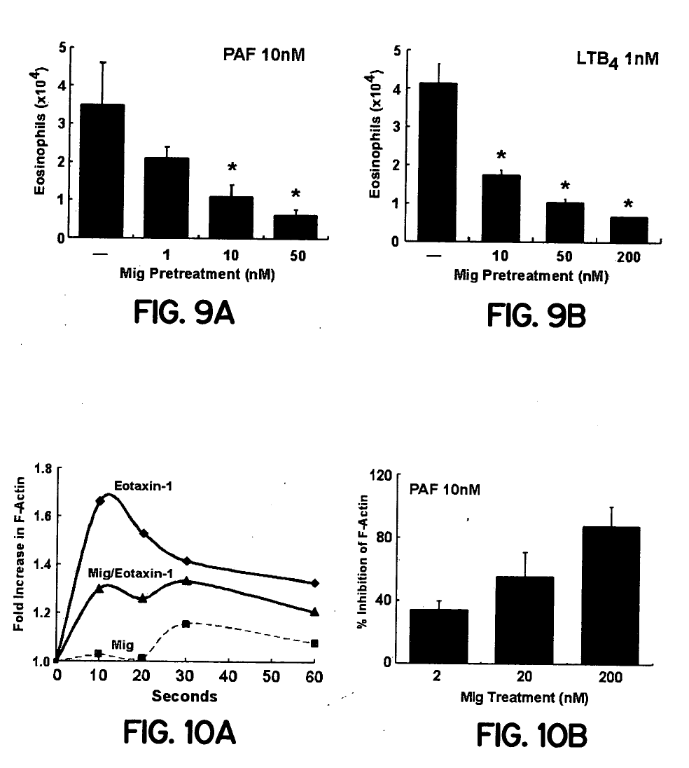

MIG inhibited PAF- and LTB₄-induced chemotaxis. It is known that MIG directly blocks eosinophil responses to the eotaxin chemokines and the ability of IL-13 to induce pulmonary eosinophilia. MIG may mediate its effect by functioning as a competitive antagonist of the eotaxin receptor CCR3. However, when eosinophils were pre-treated with MIG (FIG. 9, data represent mean±s.d. of eosinophils that migrated toward PAF 10 nM (FIG. 9A) or LTB₄ 1 nM (FIG. 9B), *=P values<0.05) their subsequent chemotactic response to the non-CCR3 ligand PAF was strongly inhibited in a dose-dependent manner (FIG. 9A). Following fifteen-minute pre-treatment with MIG at 1 nM, 10 nM, or 50 nM, eosinophil transmigration toward PAF (10 nM) was reduced 40%, 68%, and 82% respectively (FIG. 9A). The mean decrease in PAF (10 nM)-induced eosinophil responses following MIG (50 nM) pre-treatment was 75±7.3% (n=4 experiments). As a control, the mean decrease in chemotactic response toward eotaxin-1 (1 nM) following MIG pre-treatment was 91.5±4.2% (n=4 experiments). To determine if the effect of MIG was applicable to other non-CCR3 ligands, the ability of MIG pre-treatment to inhibit LTB₄-induced transmigration was examined. Following MIG pre-treatment at 10 nM, 50 nM and 200 nM, eosinophil transmigration toward leukotriene B₄ (LTB₄) (1 nM) was reduced 57%, 75%, and 84% respectively (FIG. 9B). In contrast, MIG (50 nM) pre-treatment had no effect on PAF- or LTB₄-induced neutrophil responses (17,908±4,467 vs 17,981±3,775 transmigrated neutrophils for PAF and 7,413±681 vs 6,919±591 for LTB₄; n=2 experiments). Thus, MIG had strong and specific eosinophil inhibitory activity toward both CCR3 and non-CCR3 chemoattractants. While not wishing to be bound by a particular theory, this suggested a more complex mechanism than receptor blockade alone.

Following exposure to a chemoattractant, eosinophils undergo a series of events, including reorganization of actin filaments and subsequent rapid shape changes, culminating in chemotaxis. To determine the effect of MIG on agonist-induced actin polymerization in eosinophils, filamentous actin (f-actin) formation was measured kinetically following exposure to either eotaxin-1 alone, MIG alone, or eotaxin-1 and MIG together, as shown in FIG. 10. MIG inhibited actin-induced polymerization. In FIG. 10A eosinophils were treated with 10 nM eotaxin-1 (diamonds), 40 nM MIG (squares, dashed line) or 40 nM MIG and 10 nM eotaxin-1 (triangles) for the indicated time. Cells were fixed and stained with NBD-phallacidin. Relative f-actin content was expressed as the ratio of the mean channel fluorescence between eotaxin and media-alone stimulated cells. A representative experiment is shown (n=3). FIG. 10B shows mean (±s.d.) percent inhibition of 10 nM PAF-induced f-actin polymerization in eosinophils in the presence of MIG (n=3 experiments). The analysis was performed following ten seconds of chemokine exposure. FIG. 10C shows actin localization determined by fluorescent microscopy. Cells were fixed and stained with rhodamine-labeled phalloidin after stimulation for ten seconds with buffer alone (resting), eotaxin-1 (10 nM), MIG (200 nM) or eotaxin-1 and MIG. Images were acquired with a fluorescence microscope equipped with a deconvolution system driven by Openlab software.

Eotaxin-1 (10 nM) induced a rapid and transient polymerization of actin molecules (FIG. 10A). There was a transient increase in f-actin content of 57±12% (P=0.001, n=3 experiments) within ten seconds of eotaxin-1 exposure. In the presence of MIG (40 nM), eotaxin-1-induced f-actin formation was reduced (P=0.02, FIG. 10A). MIG inhibited eotaxin-1-induced actin polymerization in a dose-dependent manner; at ten seconds, the optimal time point chosen for subsequent studies, f-actin content was inhibited 31±2% and 52±6% (P=0.02, n=3 experiments) in the presence of increasing concentrations of MIG (20 nM and 40 nM respectively). Similarly, in the presence of MIG (200 nM), eotaxin-2 (10 nM)-induced actin polymerization was reduced by 51±4% (n=2 experiments). Eosinophil treatment with MIG alone (2 nM to 200 nM) did not induce f-actin formation; the modest increase in f-actin content seen at thirty seconds (FIG. 10A) was not reproducibly detected (data not shown). Because MIG inhibited transmigration toward a non-CCR3 ligand, MIG may also inhibit actin polymerization in response PAF. To evaluate this, f-actin content was measured following exposure to PAF (10 nM) alone and to MIG (2 nM, 20 nM, and 200 nM) and PAF together. MIG inhibited PAF-induced actin polymerization in a dose-dependent manner (FIG. 10B).

Eotaxin-induced actin subcellular localization following MIG treatment was examined. The results are shown in FIG. 10C. To optimize the analysis, the maximum time point for eotaxin-induced changes in f-actin content (ten seconds) and a dose of 200 nM of MIG were used. Cell spreading and considerable cortical f-actin assembly were seen in eosinophils stimulated with 10 nM eotaxin-1. In contrast, eotaxin-1-induced cortical f-actin polymerization was impaired in the presence of 200 nM MIG. With MIG stimulation alone, the f-actin structure was markedly altered compared with untreated control or agonist stimulated eosinophils. The f-actin assembly had a punctate distribution in MIG-treated eosinophils, whereas a peri-nuclear distribution was seen in control cells. Collectively, these data demonstrated that MIG regulated actin localization; it impaired the assembly and organization of the actin cytoskeleton.

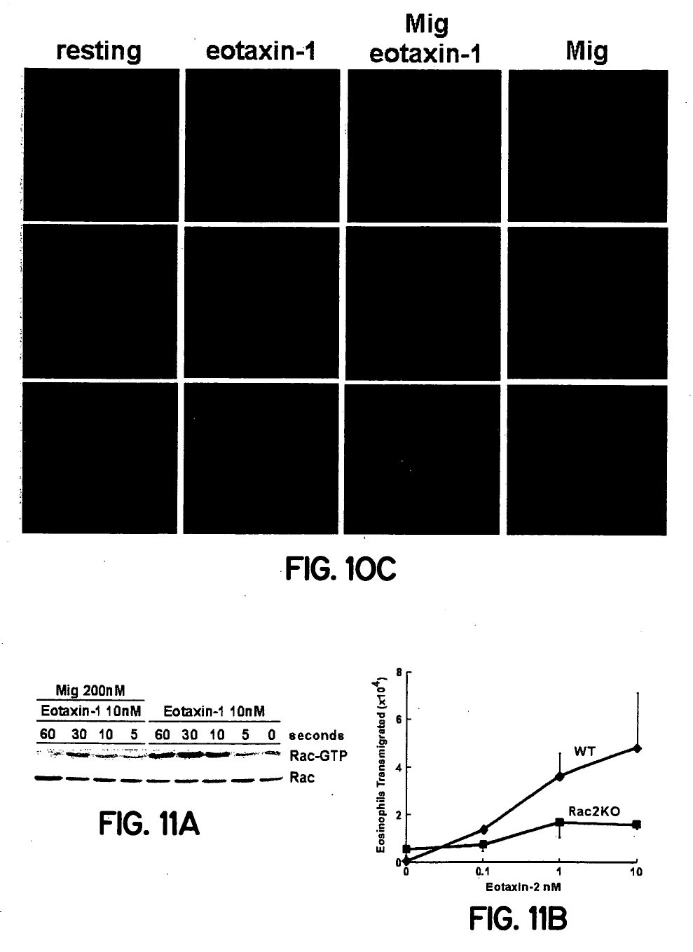

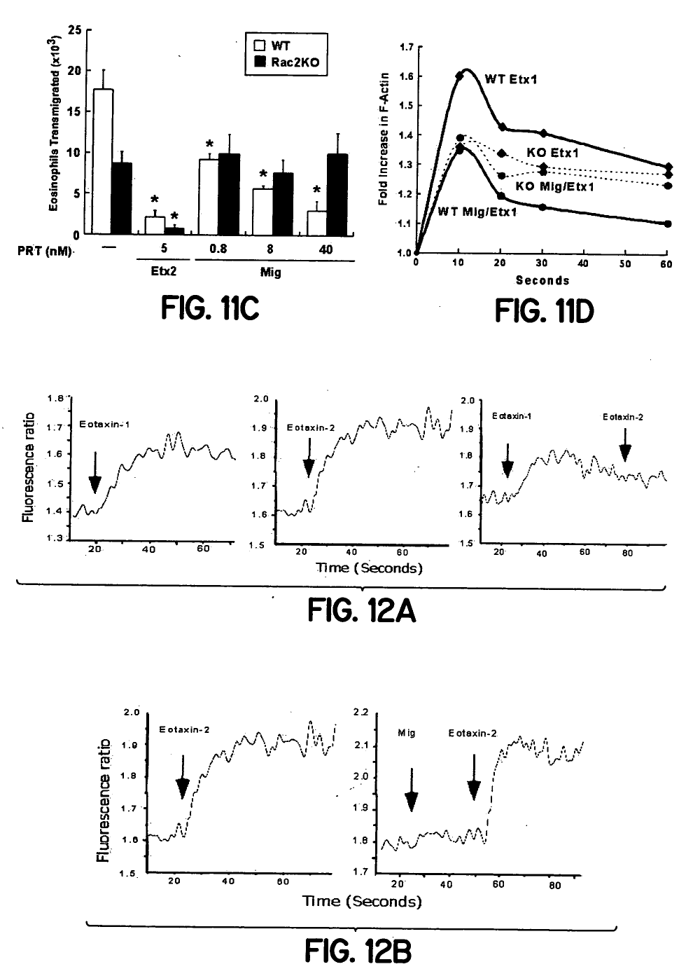

The Rho family of small GTPases have been shown to be essential regulators of cell movement and cytoskeletal rearrangement in certain cell types. In neutrophils, two Rac members, Rac1 and Rac2, direct neutrophil function, including f-actin generation, chemotaxis and NADPH oxidase activity. The role of Rac2 in eosinophil chemoattraction has not been previously determined. MIG inhibited agonist-induced Rac activation in eosinophils. Using a PAK-pull down assay and an antibody that recognized both Rac1 and Rac2, the relative levels of Rac-GTP in lysates from eotaxin-1 stimulated eosinophils with and without MIG pre-treatment were examined. As shown in FIG. 11, MIG inhibited agonist-induced Rac activation, and Rac2 was required for MIG's inhibitory activity. Eosinophil lysates were used for affinity precipitation with 5 μg PAK-PBD for 60 min at 4° C. Active Rac-GTP precipitated by PAK-PBD was separated on SDS-PAGE, transferred to nitrocellulose membrane and blotted for pan-Rac, followed by ECL detection. The results are shown in FIG. 11A. In the bottom panel, aliquots of lysates were immunoblotted and probed for Rac to confirm equal protein expression. Representative blots are shown (n=3). Only a small amount of activated Rac was detected in unstimulated eosinophils, while stimulation with eotaxin-1 induced formation of Rac-GTP within ten seconds of stimulation, with maximal levels detected at thirty seconds. MIG pre-treatment resulted in reduced formation of Rac-GTP in response to eotaxin-1 stimulation.

Neutrophils from rac2 gene-targeted mice exhibit multiple functional defects, including f-actin generation and directed cell movement. The abnormal actin localization in MIG-treated eosinophils resembled the localization seen in Rac2-deficient granulocytes (FIG. 10C). As such, Rac2-deficient eosinophils may have impaired chemotaxis and actin polymerization, similar to MIG-treated eosinophils. To test this, hypereosinophilic mice deficient in Rac2 were generated and eotaxin-induced transmigration for wild-type and Rac2-deficient eosinophils was examined. Wild-type (diamonds) and Rac2-deficient (squares) eosinophil transmigration toward eotaxin-2 is shown in FIG. 11B. Data represent mean±s.d. of eosinophils that migrated toward eotaxin-2 (0 nM to 10 nM). A representative experiment is shown (n=3). Wild-type eosinophils exhibited a strong chemotactic response to eotaxin-2, with a dose response seen between 0.1 nM-10 nM. Eotaxin-2-induced chemotaxis by Rac2-deficient eosinophils was reduced 45%-70% when compared to wild-type eosinophils. This deficiency was evident over the 100-fold concentration range for eotaxin-2. The mean decrease in eotaxin-2 (1 nM)-induced responses was 52±13% (n=3 experiments).

The effects of MIG pre-treatment on Rac2-deficient eosinophils were investigated (FIG. 11C). Wild-type (white bars) and Rac2-deficient (solid bars) eosinophil transmigration toward eotaxin-2 is shown following pretreatment with buffer, eotaxin-2 (5 nM) or MIG (0.8 nM to 40 nM). Data represent mean±s.d. of eosinophils that migrated toward eotaxin-2 (1 nM). A representative experiment is shown (n=3). *=P values<0.05 when compared to pre-treatment of buffer alone. The results are shown in FIG. 11C. Wild-type eosinophils were inhibited by MIG pre-treatment in a dose-dependent manner. In contrast, MIG pre-treatment had no inhibitory effect on Rac2 -/- eosinophils. Rac2 -/- eosinophils had a blunted chemotactic response compared with wild-type eosinophils. As a positive control, eosinophil inhibition by eotaxin-2 pre-treatment was examined. Eotaxin-2 pre-treatment desensitized eosinophils to eotaxin-2. However, in contrast to MIG, eotaxin-2-induced desensitization was independent of Rac2. For example, wild-type and Rac2 -/- eosinophils were inhibited by 93% and 92% with eotaxin-2 pre-treatment.

The role of Rac2 in actin polymerization in eosinophils was examined. As shown in FIG. 11D, wild-type (solid lines) or Rac2-deficient (dashed lines) eosinophils were treated with 12 nM eotaxin-1 (diamonds) or 40 nM MIG and 12 nM eotaxin-1 (circles) for the indicated time. Cells were fixed and stained with NBD-phallacidin. Relative f-actin content was expressed as the ratio of the mean channel fluorescence between eotaxin and media-alone stimulated cells. The results are shown in FIG. 11D. Although transient increases in f-actin were observed with eotaxin-1 (12 nM) stimulation for ten seconds in wild-type eosinophils, f-actin generation in Rac2-deficient eosinophils in response to eotaxin-1 was significantly reduced. In the presence of MIG, eotaxin-induced f-actin formation was inhibited 40% in wild-type eosinophils, but not in Rac2-deficient eosinophils. Collectively, these data suggested that Rac2 -/- eosinophils demonstrated impaired cheotaxis, Rac2 was required for normal agonist-induced chemotaxis and actin polymerization in wild-type eosinophils, and also for MIG's inhibitory activity mediated through regulation of actin assembly.

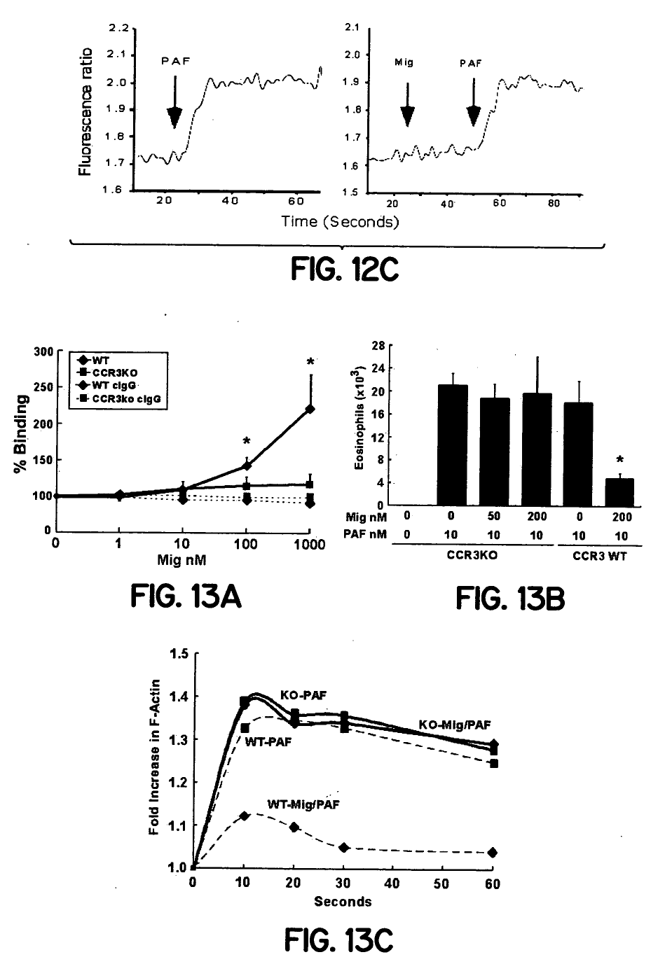

Eosinophil stimulation with a chemoattractant resulted in increased cytosolic calcium due to a combination of intracellular calcium release and an influx of extracellular calcium. The role of intracellular calcium transients in granulocyte actin polymerization and chemotaxis is unclear. The effect of MIG on ligand-induced calcium mobilization in eosinophils was examined. The results are shown in FIG. 12. MIG did not inhibit agonist-induced calcium mobilization. Calcium transients in purified murine eosinophils were measured by labeling cells with fura-2/AM, and monitoring immediately following stimulation with 50 nM eotaxin-1, 25 nM eotaxin-2 (FIG. 12A, FIG. 12B) and 10 nM PAF (FIG. 12C). Pre-stimulation with MIG (200 nM) had no effect on eotaxin- (FIG. 12B) or PAF-induced calcium mobilization (FIG. 12C). Data are representative of three experiments. Eotaxin-1 and eotaxin-2 induced intracellular calcium changes in murine eosinophils and desensitized each other (FIG. 12A); however, MIG failed to both initiate calcium flux and inhibit eotaxin-2-induced calcium mobilization (FIG. 12B). Calcium mobilization by PAF was also unaffected by pretreatment with MIG (FIG. 12C). These results demonstrated that MIG blocked specific agonist-induced signal transduction events in eosinophils that are either independent of or downstream of calcium signaling.

CCR3 was required for MIG's inhibitory activity. A single publication reported that a subset of human eosinophils expresses MIG's primary receptor CXCR3; we have not been able to detect CXCR3 on the surface of murine eosinophils. Possible CCR3 mediation of MIG's inhibitory action on eosinophils was evaluated by generating hypereosinophilic mice that were deficient in CCR3 by mating IL-5 transgenic mice with CCR3 gene-targeted mice. To determine if murine MIG directly bound to murine CCR3, MIG's binding to wild-type and CCR3-deficient eosinophils by flow cytometry was examined. Results are shown in FIG. 13A, indicating dose-dependent binding of MIG to the surface of wild-type (diamonds) and CCR3-deficient (squares) cells. Data represent mean±s.d percent mean channel fluorescence (chemokine binding) compared to no chemokine for three independent experiments combined. Staining with control antibody (dashed lines) is shown. *=P values<0.05. Dose-dependent binding of MIG to wild-type eosinophils was detected. In contrast, MIG binding to CCR3-deficient eosinophils was not significantly different from the background staining seen with the isotype-matched control antibody (FIG. 13A). These results are consistent with previous studies that have demonstrated that the human CXCR3 ligands (MIG, IP-10, and I-TAC) bind to human CCR3.

To examine whether CCR3 was required for MIG's inhibitory action, purified CCR3-deficient eosinophils were pre-treated with MIG and their chemotactic response to PAF was examined. As shown in FIG. 13B, MIG did not inhibit CCR3-deficient eosinophil chemotaxis toward PAF. Cells were allowed to transmigrate following pretreatment with buffer or MIG. Data represent mean±s.d. of eosinophils that migrated toward PAF (10 nM). *=P values<0.05. The results are representative of three experiments. While MIG pre-treatment markedly inhibited wild-type eosinophil responses toward PAF, MIG's inhibitory activity on PAF-induced chemotaxis was completely abrogated in CCR3 gene-targeted eosinophils (FIG. 13B). As shown in FIG. 13C, MIG did not inhibit PAF-induced actin polymerization in CCR3-deficient eosinophils. Wild-type (dashed line) and CCR3-deficient (solid line) eosinophils were treated with PAF (10 nM, squares) or MIG (200 nM) and PAF (diamonds) for the indicated time. Cells were fixed and stained with NBD-phallacidin. Relative F-actin content is expressed as the ratio of the mean channel fluorescence between eotaxin and media-alone stimulated cells. A representative experiment is shown (n=3). The ability of MIG (200 nM) to inhibit PAF-induced F-actin formation was lost in CCR3-deficient eosinophils (FIG. 13C). Collectively, these data established that CCR3 was required for MIG's inhibitory activity on eosinophils.

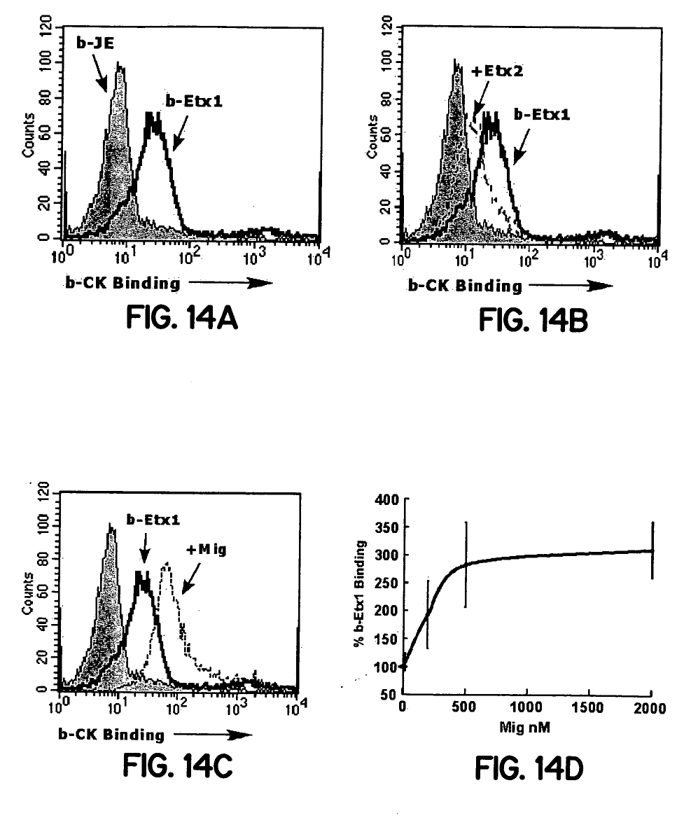

MIG altered CCR3 ligand-receptor interaction. The human CXCR3 ligands (MIG, IP-10, and I-TAC) have a distinct binding site from the eotaxins on human CCR3. To investigate the effects of MIG on eotaxin binding to murine eosinophils, binding of biotinylated eotaxin-1 to the surface of murine eosinophils was measured by flow cytometry. Binding of 10 nM biotinylated eotaxin-1 (b-Etx1, black line) without (FIG. 14A) or with 200 nM eotaxin-2 (FIG. 14B) to the surface of eosinophils compared to control chemokine (biotinylated JE, b-JE, filled histogram) is shown (representative histogram from 3 experiments). Binding of eotaxin-1 (black line) with or without 500 nM MIG (dashed line) is shown (FIG. 14C)(representative histogram (n=3)). FIG. 14D shows percent eotaxin-1 binding in the presence of 20 nM-2000 nM MIG compared to eotaxin-1 alone. Data represent mean±s.d. of mean channel fluorescence of eotaxin-1 binding in the presence of MIG compared to eotaxin-1 alone (n=3 experiments).

There was specific binding of eotaxin-1 (20 nM) to the surface of eosinophils when compared to binding of a control chemokine JE/CCL2 (20 nM) (FIG. 14A). Incubation with 10-fold excess unlabeled eotaxin-2 resulted in reduced binding of eotaxin-1 (FIG. 14B). Incubation with MIG (500 nM) resulted in markedly increased binding of eotaxin-1 to the surface of murine eosinophils (FIG. 14C). Following incubation with MIG at 20 nM, 200 nM, 500 nM and 2000 nM, there was dose-dependent and saturable binding of eotaxin-1 (FIG. 14D). Together with the actin/Rac2 data, these results support a model where MIG induces a conformation change in CCR3. This CCR3 conformational change initiates an inhibitory signal transduction cascade, resulting in reduced Rac activation, altered actin organization and assembly, and inhibition of agonist-induced chemotaxis.

The eosinophil-specific inhibitory activity of the natural chemokine antagonist MIG was determined. MIG potently inhibited eosinophil transmigration toward both CCR3 and non-CCR3 ligands, demonstrating activity beyond receptor antagonism alone. MIG initiated an inhibitory signaling pathway that resulted in reduced agonist-induced Rac activation and impaired actin polymerization and localization. MIG treatment alone resulted in abnormal actin organization, demonstrating a prominent role for MIG in regulating cytoskeletal rearrangement in murine eosinophils. The impaired actin localization resembled that seen in Rac2-deficient granulocytes. Consistent with this observation, Rac2 was required for optimal CCR3-dependent chemoattraction and for MIG-induced inhibition. Rac2 was not required for ligand-induced homologous desensitization of CCR3, thus CCR3 ligand-induced desensitization and MIG-induced inhibition were mediated by distinct mechanisms. The inhibitory activity of MIG occurred independently of calcium mobilization. This finding indicates that inhibition of calcium flux should not be used to screen for the presence of chemokine receptor inhibition because a powerful inhibitor like MIG can induce profound effects independent of calcium signaling. Using CCR3 gene-targeted cells, a role for CCR3 in mediating MIG's inhibitory activity was elucidated. Collectively, these data indicated that MIG generated negative or inhibitory signaling via CCR3. CCR3 was previously regarded solely as a powerful activating receptor in eosinophils. Thus, depending upon which ligand engages CCR3, a completely different signaling cascade is engaged.

The mechanism by which an activating and inhibitory chemokine resulted in opposing activity on eosinophils utilizing the same receptor was evaluated. A model is proposed in which binding of MIG to CCR3 promotes alterations in the receptor that are associated with initiation of negative signaling. In particular, MIG inhibited agonist-induced Rac activation and disorganization of the f-actin cytoskeleton, which promote cellular inhibition to multiple stimuli. In contrast, engagement of CCR3 with activating ligands (eotaxins) induced a marked calcium flux, activation of Rac, and F-actin reorganization and polymerization. A MIG induced conformational change in CCR3 that induces inhibitory signaling is supported by MIG's increased eotaxin-1 binding to the surface of murine eosinophils without changing the surface expression of CCR3, presumably by inducing a receptor conformation that altered agonist-receptor interaction.

Rac2 deficiency in eosinophils resulted in defects in directed cell movement, a function requiring actin cytoskeletal rearrangements. The data demonstrated a regulatory role for Rac2 in actin remodeling in eosinophils. Rac2 was required not only for optimal actin polymerization and chemotaxis in eosinophils in response to an agonist, but also for MIG's inhibitory activity, supporting the concept that Rac2 is a key regulator of eosinophil chemoattraction. MIG-induced inhibitory signaling may be promoted by the interaction of GTPases with guanine-nucleotide exchange factors (GEFs) that catalyze the GDP-GTP exchange reaction. GEFs contain pleckstrin homology domains and are regulated by the products and substrates of PI3K. MIG may control Rac activation by regulating GEF or PI3K activity.

These data support a new paradigm concerning eosinophil chemokine pathways by demonstrating that eosinophil chemokines induced diverse signaling pathways. The major eosinophil chemokine receptor CCR3, previously identified solely as a powerful activating receptor in eosinophils, can transmit positive or negative signals depending upon the ligand engaged.

The action of the chemokine CXCL9 (MIG) on eosinophil chemoattraction and function extends beyond receptor antagonism alone. In addition to inhibiting eotaxin-induced f-actin formation and chemoattraction, MIG blocked PAF- and LTB₄-induced responses. MIG-treated eosinophils displayed an abnormal f-actin assembly in the absence of agonist stimulation. MIG pre-treatment inhibition of eotaxin-induced activation of the Rho-GTPase Rac and Rac2-deficient eosinophils demonstrated an impaired transmigration and actin polymerization response to eotaxin stimulation. MIG was unable to inhibit eotaxin-induced responses in Rac2-deficient eosinophils. Using CCR3 gene-targeted cells, MIG's inhibitory activity was demonstrated to be mediated by CCR3. Thus, by altering agonist-induced signaling and abrogating cytoskeletal reorganization by a Rac2-dependent mechanism, MIG inhibited eosinophil responses to diverse stimuli. These results established that distinct chemokines utilized CCR3 to induce opposing signals in eosinophils.

A MIG composition may be administered to a mammal, such as a human, either prophylactically or in response to a specific condition or disease. For example, the composition may be administered to a patient with asthmatic symptoms and/or allergic symptoms. The composition may be administered non-systemically such as by inhalation, aerosol, drops, etc.; systemically by an enteral or parenteral route, including but not limited to intravenous injection, subcutaneous injection, intramuscular injection, intraperitoneal injection, oral administration in a solid or liquid form (tablets (chewable, dissolvable, etc.), capsules (hard or soft gel), pills, syrups, elixirs, emulsions, suspensions, etc.). As known to one skilled in the art, the composition may contain excipients, including but not limited to pharmaceutically acceptable buffers, emulsifiers, surfactants, electrolytes such as sodium chloride; enteral formulations may contain thixotropic agents, flavoring agents, and other ingredients for enhancing organoleptic qualities.

The dose of MIG administered in the composition to a mammal is in the range between about 10 μg/kg to about 10 mg/kg. The dose of IP-10 administered to a mammal is in the range between about 10 μg/kg to about 10 mg/kg. In one embodiment, a dose of about 30 μg/kg of MIG or IP-10 is administered. Dosing may be dependent upon the route of administration. As examples, an intravenous administration may be continuous or non-continuous; injections may be administered at convenient intervals such as daily, weekly, monthly, etc.; enteral formulations may be administered once a day, twice a day, etc. Instructions for administration may be according to a defined dosing schedule, or an “as needed” basis.

Other variations or embodiments of the invention will also be apparent to one of ordinary skill in the art from the above figures and descriptions. For example, linear peptides with homology to MIG and/or IP-10, exhibiting a similar functional activity as MIG and/or IP-10 (analogues of MIG and/or IP-10), may also be administered. Thus, the forgoing embodiments are not to be construed as limiting the scope of this invention.

1. A method of perturbing eosinophil recruitment or function via a CCR3 eosinophil receptor, comprising (i) selecting a MIG ligand to bind the CCR3 receptor to selectively regulate a negative signaling pathway in the eosinophil, or (ii) selecting an eotaxin ligand to bind the CCR3 receptor to selectively regulate a positive signaling pathway in the eosinophil.

2. The method of claim 1 wherein the negative signaling pathway is at least one of inhibited eosinophil transmigration, inhibited Rac activation, or impaired actin reorganization.

3. A method of preferentially regulating a CCR3 mediated chemokine signaling pathway in an eosinophil, comprising (i) selecting a MIG CCR3 ligand to preferentially induce a negative signaling pathway, or (ii) selecting an eotaxin CCR3 ligand to preferentially induce a positive signaling pathway.

4. A method of perturbing eosinophil recruitment or function via a CCR3 eosinophil receptor comprising providing a ligand for the CCR3 eosinophil receptor to selectively regulate either a positive signaling pathway or a negative signaling pathway.

5. The method of claim 4 wherein the ligand is an activating chemokine, an inhibitory chemokine, or combinations thereof.

6. A method of selectively inducing signaling via a CCR3 receptor comprising providing a pharmaceutically acceptable composition comprising MIG to the CCR3 receptor thereby inducing a conformational change in the CCR3 receptor that selectively induces an inhibitory signaling pathway.

7. A method of perturbing eosinophil recruitment or function via a CCR3 eosinophil receptor comprising providing a ligand to bind the CCR3 receptor to selectively regulate a negative signaling pathway in the eosinophil.

8. The method of claim 7 wherein the ligand is MIG.

9. The method of claim 7 wherein the ligand is a synthetic small molecule.
